SEQUENCE LISTING (56 sequences)

SEQ ID NO: 1 - DNA, 910 bp, Homo sapiens
ccagcgtcaggccagccccggcatgctctgcggccgcccgcggtccagctccgacaacaggaattttctccgagagcgggccgggctcagttcagctgctgtccagacccggatcggcaacagtgccgcctccagacgttctcctgccgctcgcccgcccgtcccagcgcccccagccctcccgcgagggcgccccgggacggaaggatccaccagtctgtcggcgcccgccgttctcgtggtcgccgtcgccgtcgtcgtggtggtagtctccgccgtcgcctgggccatggccaattacatccacgtccctcccggctccccggaggtgcccaagctgaacgtcaccgttcaggatcaggaggagcatcgctgccgggagggggccctgagcctcctgcaacacctgcggcctcactgggacccccaggaggtgaccctgcagctcttcacagatggaatcacaaataaacttattggctgttacgtgggaaacaccatggaggatgtagtcctggtgagaatttatggcaataagactgagttattagtcgatcgagatgaggaagtaaagagttttcgagtgttgcaggctcatgggtgtgcaccacaactctactgtaccttcaataatggactatgctatgaatttatacaaggagaagcactggatccaaagcatgtctgcaacccagccattttcagtttatcatcgttgactctttgcaaaggaaaaactacaagatgttttggattaaccggctgcagagggtcaaggcttctgcttagttttttctagttagtgtttgagttgaagtgttatacatttcttactgccgattgcttattacttaattgttcatgttttcagagttcttgcctattaaaattttattaatttggttaacaa

SEQ ID NO: 2 - DNA, 7101 bp, Homo sapiens
ccagcgtcaggccagccccggcatgctctgcggccgcccgcggtccagctccgacaacaggaattttctccgagagcgggccgggctcagttcagctgctgtccagacccggatcggcaacagtgccgcctccagacgttctcctgccgctcgcccgcccgtcccagcgcccccagccctcccgcgagggcgccccgggacggaaggatccaccagtctgtcggcgcccgccgttctcgtggtcgccgtcgccgtcgtcgtggtggtagtctccgccgtcgcctgggccatggccaattacatccacgtccctcccggctccccggaggtgcccaagctgaacgtcaccgttcaggatcaggaggagcatcgctgccgggagggggccctgagcctcctgcaacacctgcggcctcactgggacccccaggaggtgaccctgcagctcttcacagatggaatcacaaataaacttattggctgttacgtgggaaacaccatggaggatgtagtcctggtgagaatttatggcaataagactgagttattagtcgatcgagatgaggaagtaaagagttttcgagtgttgcaggctcatgggtgtgcaccacaactctactgtaccttcaataatggactatgctatgaatttatacaaggagaagcactggatccaaagcatgtctgcaacccagccattttcaggctaatagctcgtcagcttgctaaaatccatgctattcatgcacacaatggctggatccccaaatctaatctttggctaaagatgggaaagtatttctctctcattcccacaggatttgcagatgaagacattaataaaaggttcctaagtgatatcccaagctctcagattctccaggaagagatgacttggatgaaggagattctttccaacctgggctcacctgttgtgctttgccataatgacctattgtgtaagaatataatctacaatgagaaacaaggtgatgtacagttcattgattatgaatattctggatacaactacctggcatatgatattggaaatcatttcaatgaatttgcaggtgtgagtgatgtagactatagtctgtatccagatagagaactacagagtcagtggctgcgtgcttaccttgaagcctacaaagaatttaagggctttgggactgaagttactgaaaaggaggtagaaatactcttcattcaagtcaatcagtttgcattggcttctcatttcttttggggattgtgggctttgattcaagccaaatactccactattgagtttgatttccttgggtatgcaattgttcgttttaaccagtactttaaaatgaagcctgaggttactgcattaaaagtgcctgagtaaagaagagatttaattattctccagtagctgagcaatgcttgtgaatcttttcttaagaaatcccaaaaagccaatattagttaaaattctgttgtttaatttggttatcttgctttataaattatgcctctaaacaatcaaatctatttttgaaatagactgaatgatgtcaagaaatatacctactgctatccgtatgtggtggattagaaatgtgttaaatctgcaaaaggtataaagatgtcagtttaatttctttgataatttaacctatgttgtatgtgaattatttattataaacttagcacgattctgtgactgtttttctctgtttcacgttcgttgagtgtaagcaatgaaaatgtcccaaataagttttttaagttttactttaataagattaatttcagtaaacattctagttgttcagtgtaacctttttatcttgatgcattgtaagtaaaatgaatcatttactcttgaaatgccagtcattgactgatgtagataatttaggattttcatataaaaatagctgtttaggaaggtgaaatacattcactgtctctgttggtggtacatcttgttgaattcaatattagaaagtatttctttttggggtaatataacttagaattaaatccctgtttctctatgtagtctggcagtataaatataaatatttaccatataatcttggaataagtattagttaatgttaccaaaatctgtattaaataatgttttcaaatgctaaatatggtcgttactattttcagttttaaaaattttatagtatcaaattgtttctaaccaaaaatttccttttacttctagagatgctttatgtttttgattattaaatagtcactagtattgctaatttttgaaacaaatacacaaaatttatcatatgtagaaataagaaaatattgtttaggttaattattttttacctatccagagtcattccttacatttcagtttcatgtttcattcctcacattttagtttcatgttttagtttgattctatgtttttagactgatagcaaatgattacttgatacatgtaagttcagcgttatatgttgaggcagttatataattaaataaggaacacttagggcattgatcttgtacacttaaaatctgacaaatgacaaaatgtgaattaagcataaatagtcatggcaggagggcttttgtttatttctcttgtttttgtttaactgtatttttaattttgtctggtggtaagagggaggtaattattgtatggaaagaagttagatctttcagatagataaataggcttcaggttttgaagtctgctctgtgtagttgagcagtcatggttaactttacttagaaaattagtctgacaagctttgcacttcggtcacataagtgcccatgtacaaagttggcttactggatctgcatattcaaaatatgcaactacaaatttatcctctgagaaatcttctgaggagtctaatgtattccaaatgacttaatattctcagacaaatgctttaaaatacttggattatctcaagatttttgaagctaataaagtaaatagaaacacaggccattttaaatggtaaattctagttacataaatataatcttaaagtatgtttgtatctgtaacagtgttagaccagatttggtttctacctccccaatgttataaatgttcacttttgtttttataaaccatattactgtaagatcaatagaactttgcctagttaagtgaatggaaccaattaaacaactagtagctttttataatgttagtgtgaacttgaaaaattgtttttggtggaatttttgtctgtggttagaacattttcataaaacagataaagaatgtgtaatactcttaattttttttttttttttttttttttttttgctttctgccttctaaagtgcaacagtatatatcttgtagaactgtgggaggagagaagataatttttctaccttactccatattcttacttcaaactattttgaatagaggttcactgagccattgctgatagactgtgcatagctactagctgtcacactattgtaggttttttggagagattcatagggaagtgaatgtaaccaggaataaaggctctcactttagctctgggactatgcagtggatcttgggaacaaaataagatatctaatctattttccccattctggctagattttggttgttgtgatgctcctacttcctgtataggtttttgggatcatggtgcaggctagtaataggattcagaattgcttcccttttatatcgaccatgtaagggattctaaaggagggtaaatctcaaagtgctctgaaatcatccctctaaaatctgtcctgtggtgtgctaagatgtctgttgcctttttatgttaagctaaggaacttgaatgccttacctaataactcaactagtagtaagctcattttgtaaatatgaaaatggctgtaagaagtggcattcatgtatattcaatgttagtcaatagaccatagtggcctggatgctttaacaagccaaattccgcaattttttttccttttttccccttcatacttgtatgatctcacctgaccagtgatttcttttcctcctaaccccatgctaccaccattttctggaaatcaaagagatgtggtagtaaatcatacagtacacacgatcatctgagatcaatatgagcacaaaactcatcaagtaggtctgcctggaatgtatataaaaaatgtaaataaaaatttgtaaattagtgtgaactgtatcttgcatatgagactgtgtattgttttgcattctcttcccttttgtagaaatttttctgtaaggaaatgattcagcttgttggttttagagtaaaaatagccaacagttgtggctacacagtgagatcacaggaactggaaatagggtttgtcggctttggttaatgtcagtactcatttatttagcctctcagtgcttaataagacttttctttattttttagttagccgttaggttttgtgaggttagattcctggaagcagtgaatttatacactgttatattaatagagctccgttctttgagtcatgattccaagctaaaggaaattaaaaatgtaatttaataatttcctatttttagggttgttaatttttttctacaaaaaaaccttgaaattttagatatcccaatgtgaatctaatttccatatatacagaaattagacaaataataagtctttagttcaacttaagcatatctcaaatgacttctctaaatttaaagttgatcatgataggatcataaaagacagaaaagacttaagtaatcttgtaatgacaattatttccatttttgctgaactaaaaatatttaacttcataaatatgttactacagcttccagatttaaagaaaaaaagtttcccccactctcaattaaaagttagaaccctccacttttaaaattatacaaatatttcttttttacattacacagaagccttctgtaccattttacgaatttctgtcttcataatataagtgaaaatactgtcatttcaattttctgctttaaattgtttttaataagcattccaaagtgatacagacttaagcttttaatcaatcagtcattcagttgatagacaaagttagcgatgctttatgctaggaaacttgttgacagtaacctgtgcgactttatgcagaagacaaatgctagtaattattatgcacagaggaaaaatcattttaagtatgtggtaaagcagcttcatctttcaaaattgatttgctctggtttttctttagtccattagattccagaatgtccttttactgggaatttagttatgtattaagataacctgttttcagttctttttgaaaagaagacattatttatattgaaccaccttattttaaaatttttaacttttatataccacttgtgtgattccagtgtcatgtcttgggtttgatgtcgttggacagaaaagtgtatcaattattttaaatgaatttttccccatgtttgaggcttagtctgtaaatgtgttgctgtaacagaaaatacttgggtatgcattacttgaatacttgaaaactgaaattaataagatgtattacataatgaattagatttctctgaacagtttttacactgaaaatcttcatttctggattgcagtttgaaatggaatgaagacctgaattatttgggtagaaaaaattatgatagtgcttataagaactgtaaactgttttaaactattttgtgtttgacgcatcaaacttcaagttttttgtaagtttctctcctgaaattttctttctcttctatactttatgcacttactatactactgatgtaataaaagagcagggttaaaaatattgtatctgtattcattgtgaatcctgtagcttttctagttaacaaaaaatcgctttctaaaatactcttaatcccattgttttggttaacatcttacccatttgttgtatttcaaatgccattaatcattttagtacaacacctatgtttataaaaatttgaaaacattacatattgtatttaaaactaattagtgaagagtaagaaaaaaactagccaacagaattgtaggtgatgcattagttaaatttcaaaactcataataaaggaactttcagagattggttgaaacccagtggtatccctgtaaattagctcctgtgactggaaaagaccccaaaaaggcagtagaggagattagtgtttacttgctgtggttgtggtgtgctgctacttaattataggtagtgacacactgaaattcttatttgtccaataatctgaagtagtttcctatatttatctgtactaaattgactataaattgagtctgcaaagaggaaactttttgactgtactgtatttaggagcctttgtacagcttggtcaaatttccatgatatgaagtatttgagttttaaaatatactgttattaaaaggaaaaagacatggccattattccatgtgcttaaatgataatttccttattcagtttcagaagaaaaagaatgaaattgggtaactgtcattgcgttagctttatgttgaattgggaaattgtggcataaagcttaaattcgtgtttatcaaatgtgaaccatagtagtataatgctgctttgtatataatgtaagtgctacaaatagtctcagcactgaaaatgtattgatacctcttaaatgaatgcaacttttgatgtaggtgttttgctatgcctcagaaaatatctgtctgagaatttgttaatctgtttgataatgaagatacttcctgttttcttgtttcatattttcatgttcaaaatttaagttttacatttttactactgttaatttaaataaaatttgttctgtggataaaatgaggttggcagtgaagaaaattaaaaacagcctcattcatgtaactggttaagtaaaaatacattttcactatgtgttcataaacttttaatgaagctgtttgtctttcagttcaaatataagtgatgtttaggctttatttctgttaataaggctttttaccattgattaaatgaaggaatgtatctttttgaagagatttatattctgtaaataaaaattcgttgtaacaataaagttgagttctaactacagtgtattca

SEQ ID NO: 3 - DNA, 2434 bp, Homo sapiens
tgaccaagaggaacagaacacagagcagcctggcagtgtccaagcaacaagcctccgctcctccttcctgcaccctggggctcctgaaactcacatgggtaaaaaagatacagtaaagacataaataccacatttgacaaatggaaaaaaaggagtgtccagaaaagagtagcagcagtgaggaagagctgccgagacgtggatccagtaggaacatagatgcatccaaactcattagactacaaggatcacggaaactgttggttgacaatagcatacgggaactgcaatacacaaaaactggaattttttttcaggctgaggcctgtgtgacaaatgatacggtatacagggagctaccctgtgtttctgagaccctttgtgacatctcacattttttccaagaagatgatgagacagaggcagagccattattgttccgtgctgttcctgagtgtcaactatctgggggggacattcccaggtaagaaaagctattccgttggaaaactattggacgctgtgtctcaatatcacagaggaacatgagaattagaggaatttgggtgtttttcatcgaactcaggggagaatgaatgagcttatgcaatactagcaatgaatccagaaatttttgaaacaaaccttaagcaatgaggttttcttccatggaggtgtctagagaacctatacacaaaaaggcaatgagcaatgcaatcgaagaccagtgggttattggttaagctgttactcaatacataaaaccaacctaattctctaggaaactggctgatgatgaggctgaaggccacagtggggtcctgaggtttaacaccggggaggggagaatgcgtctatttgtagggtatgccaatttccaagggttgaatatccccatcatggcagatttcaaactaccaacgtgaagcccctgatgagggagttgaaagaacacatgcagaagtctgtcatcctgctttcaccacacagatacaagagacataaataacctcaagggcatagactgctgaaaagtgtagaaaaatgactggaaattggtatgttttaatatgtattacttttgtttttaatatgatttatttaaatgttagcttgtatcatttaatttagactaatggttgtgttgaacaacttggttgcaaaagccctgaaaatggaaccgtcagctctcatgatctgggatgcgctgtctgcagttcagcgctgggttcctcccactttctaaacccctcctcagcaggcagactccagcatgcacatgcaccaggaaagagaatataaaatggcatgtttactcttgctgctttgcctgtagctacccgaatccatagcaaaatgtgtagccgcagacagcaaccagtcttaggatgtcagcccaggggtctctccatgcttggcttgcccacagaatccttccgagaaaagcgctgtaaagcctttctcttcatcccacttctctaggtacctgttctttttaaacctaatccacgtgtcagtttctctgctggtctctttcacgtcgaaataaacagatatctcaaggaatggatcaaaggcagagaatttgatggagttggttgccttgctccagaaggtatcacctgtgttagggcaggtttctctccagaggcatgacgaaccattggctcacgctgtatgtcatagaacccatattggctaatattggaatcacctgtgaggctttgaaagcgccgatgctttggtgccacctccagacttctgattcaattgatgtgggctgtgacctggtattgggtggtttttaaagcttcccaggtgtggtcaagtttggaattaccttggaatataaatgcatacattttacgcattggtgaattactgcttcagtctttgattccgacatcgtatgataccaatcggagcaatcaatttaagccagagaatctggctgtgtccacattgaacgtgggcataatacaaagagaagaaggagagatgtcaagacacatttggctcaagggagtgtgttatctctattatttataaatgtggataaaaatatcttcattcaaaatgtcttctatttcttgatagtctagatccgcccagtctaagagaactttctgtgatgacgggagtgttccattctctgctacacagtgttatagccgtgagtgatatgtagctgttgagcacttgaaatatggatagtgcaactgagaaactaaagtttacctaagtttaattcatacaaatttaaatagcatatccgggtagcggctactctacgggacagtgcagctctagttttcaataacatcgtcttcatcttcatcaactgattcttgatgtttttcatctttttgtaacatggggatcccaacctccagtgtatcagaagagcaggaatcttcagagggacaagattcag

SEQ ID NO: 4 - DNA, 5198 bp, Homo sapiens
agaaggagggctgtctgagattcgagggaaacaagctctcaggacttccggtcgccatgatggctgtgggcggtaaacgcggttagtgcaagcatctgggccatcttcaatggtaaaaaagatacagtaaagacataaataccacatttgacaaatggaaaaaaaggagtgtccagaaaagagtagcagcagtgaggaagagctgccgagacgggtatacagggagctaccctgtgtttctgagaccctttgtgacatctcacattttttccaagaagatgatgagacagaggcagagccattattgttccgtgctgttcctgagtgtcaactatctgggggggacattcccaggagacatttgctcagaagagaatcaaatagtttcctcttatgcttctaaagtctgttttgagatcgaagaagattataaaaatcgtcagtttctggggcctgaaggaaatgtggatgttgagttgattgataagagcacaaacagatacagcgtttggttccccactgctggctggtatctgtggtcagccacaggcctcggcttcctggtaagggatgaggtcacagtgacgattgcgtttggttcctggagtcagcacctggccctggacctgcagcaccatgaacagtggctggtgggcggccccttgtttgatgtcactgcagagccagaggaggctgtcgccgaaatccacctcccccacttcatctccctccaaggtgaggtggacgtctcctggtttctcgttgcccattttaagaatgaagggatggtcctggagcatccagcccgggtggagcctttctatgctgtcctggaaagccccagcttctctctgatgggcatcctgctgcggatcgccagtgggactcgcctctccatccccatcacttccaacacattgatctattatcacccccaccccgaagatattaagttccacttgtaccttgtccccagcgacgccttgctaacaaaggcgatagatgatgaggaagatcgcttccatggtgtgcgcctgcagacttcgcccccaatggaacccctgaactttggttccagttatattgtgtctaattctgctaacctgaaagtaatgcccaaggagttgaaattgtcctacaggagccctggagaaattcagcacttctcaaaattctatgctgggcagatgaaggaacccattcaacttgagattactgaaaaaagacatgggactttggtgtgggatactgaggtgaagccagtggatctccagcttgtagctgcatcagcccctcctcctttctcaggtgcagcctttgtgaaggagaaccaccggcaactccaagccaggatgggggacctgaaaggggtgctcgatgatctccaagacaatgaggttcttactgagaatgagaaggagctggtggagcaggaaaagacacggcagagcaagaatgaggccttgctgagcatggtggagaagaaaggggacctggccctggacgtgctcttcagaagcattagtgaaagggacccttacctcgtgtcctatcttagacagcagaatttgtaaaatgagtcagttaggtagtctggaagagagaatccagcgttctcattggaaatggataaacagaaatgtgatcattgatttcagtgttcaagacagaagaagactgggtaacatctatcacacaggctttcaggacagacttgtaacctggcatgtacctattgactgtatcctcatgcattttcctcaagaatgtctgaagaaggtagtaatattccttttaaattttttccaaccattgcttgatatatcactattttatccattgacatgattcttgaagacccaggataaaggacatccggataggtgtgtttatgaaggatggggcctggaaaggcaacttttcctgattaatgtgaaaaataattcctatggacactccgtttgaagtatcaccttctcataactaaaagcagaaaagctaacaaaagcttctcagctgaggacactcaaggcatacatgatgacagtcttttttttttttgtatgttaggactttaacactttatctatggctactgttattagaacaatgtaaatgtatttgctgaaagagagcacaaaaatgggagaaaatgcaaacatgagcagaaaatattttcccactggtgtgtagcctgctacaaggagttgttgggttaaatgttcatggtcaactccaaggaatactgagatgaaatgtggtaaatcaactccacagaaccaccaaaaagaaaatgagggtaattcagcttattctgagacagacattcctggcaatgtaccatacaaaaaataagccaactctgacatttggattctaccatagactctgtcattttgtagccatttcagctgtcttttgattaatgttttcgtggcacacatatttccatccttttatgtttaatctgtttaaaacaagttcctagtagacaccatctggttgagtcagttttttttatggtgtattttgaacccattctgatagtctcttttaactggaagatttcaattacttacgttaatgtaattattaatatgttaggatttatcctcagtcagccagtttgttatgtcttttctattctactgttatcacatttgtaccacttaaagtggaatctaggcactttatcaccatttagatcctattaccttttctcatctaggatatagttatcttctacataatctttctgtatcttaaaacccatcaataaattattatatattttctacttttaatcactcagaagatttaaaaaactcatgagaagagtaatctgttatgtttttccagatatttaccatttctgttgctcttccttcattattttccaaatttcgttctgcaaatttccacttcctctgatagacgttttttagttcttttagagtggttctgataggtacagattctcttattttttgcttcctctgaggacatctttttctcaccttcattctcagtgatgttttttgcttgtagtatttttagttgacattgttttctgttcagcagtttccttttagcttccgtatttcctgatgagaaatctgcagtcattcaaattgttgtttccctgtatgtagtgtgtcatttttctgtcagatttcaaggtatttatctttagtttttagccatttcattatgttggggatgagtttccttgttttattccctttggaatttgctccaattcataaatttgcagttttatgtcttttaccaaacttagaggttttcagcctaatttctaaaaatactttttattagcctgattttcatctttataggaaatagtttaagtgatgacaagttccaatagcttatatgcccagaaggccttcaaaataagaattttgaaagaatacagaaaacaaacttttatatccttctcatgtcttctactgtaaaattcatatgctttgctactctaaacctagtttgaaatcaacagtcttgagaatagatgaaaattttgatgaatagtggaattcttttaaatggaaacctcttacatgtgattttccttgccatctagaaataaaccatagtatttatgttgaatcaatcaatattatattttgtttttttcctcctcttctgagactcttattgtggaaatgttagacttttatgttttcctaaatgtccctgatattctacttatttagaacatcttttcattttttccattattctgattgggtaattttaatttgtctattttcaaatttgctggagtgttcacctgttgttatctgtgtcgtcccactgagtgcattcaccaccttttaaattttggtcactgtatgtatcagttctaaaatttccattttgttctctatattttaaatttcttgccttatattctattttcctgcaaatgtgtcagcatttgcttgtttgagctttttttttttcaagacagggtctcaactctgttacccaggctggagtgcagtggtgcgatctcagctcactgcaacctctgcctcctggttcaagcgattattgtgcctcagcctccagagtagctgggattacaggcatgcaccaccacagcccagctaattttttgtatttttagtagagacagagttttgctatgttggccaggctggttttgaactcctggcctcaagtgatccacccacctcagcctcccaaagtgctgggattacaggccactacacctggcacatttgagtatttttttttttttttttttttttttgagatggagtctcgctctgtcatctaggctggagtgcagtggtgtgatctcagctcactgcagcctctgtctcccgggctcaagcgattctcttgcctcagcctcctgagtagctaggactacaggtgcatgccaacacgcccggctaatttttttttaaaaatatttttagtagagacagggtttcaccattttggccaggatggtcttgatctcctgacctcatgatccacccgcctcggccttccaaagtgctgggattacaggcatgagccaccgtgcctggcctcatttgagtatttttataatgtctcttttaaagtctttgtcagataatttcactgtacatgttattcagtgtttggtgtccactgagttgtcatttgccagacaagtggagatttttgcagctcatccttgtattctcagtagttccgatatgtaccctcgaaatgtgaatgttatcttatgagactctgttttatttgtatccaacagaagatgtttattatttatttggctttctgtgaactgaggtcttaatatcagctcattttaaaagtctttgcagtggtattcggatctatcctgtgtgtgcctatgagattgggtgcagtgtatcctgttagctccattctcagggcgtttgaatgtgaattaggaccagcgcaatgaatgctcaagttggggttgggcgttagaattcataaaagtctttatatgctcagattctgcagctccatcctcttcatgattgtgatcgtaaggacagtggaattttgcagttgtctataccttatcttgaagtagaactaaattgttacatttgaagaatgtacttcataaatttacaaaaataaaaaaataggttaatgttattaaaacaaaaaggcacaca

SEQ ID NO: 5 - DNA, 2359 bp, Homo sapiens
aaatctgtccgcccccttggccactgatcccccgaagagcttctgtcgccgctctaggaatagagacattgaagtttgggacaagatatttatctaacttctgtgtcaaaattagcgacctgctatggcaatgaagaaagaaactgaatttgtcattttcacctgaagaaaaatgatagacaaaaatcaaacctgtggtgtaggacaggattctgtgccctatatgatttgtctgattcacatactcgaagaatggtttggtgtggagcagttggaggactatttgaattttgcaaactatctcttgtgggtttttacaccactaatacttttaatacttccttactttactatctttcttctctaccttactattattttcttacacatttataagagaaagaatgtattgaaagaagcctactctcataatttatgggatggtgcaaggaaaacagtggcaactctgtgggatggacatgcagccgtttggcatggtaagcaaggatactttcacctttgtgttgccattcatgtgtgctgcattggaactgtgttaccattccattttattgattgagaagatactgaaaaatataatagaagtgatatttactattccctgttacctgtttgcatgcattgtgcatatgtatgtgtctttcttaaaatgagttttcccatagacgttggtttccattaaacaaagaaccacgcagcactgtagactataagttgaaataagaaaatttatatttaaaacatctaaaagagtaagagactaaataaagctgtagaaaactgctttaaaaatgcttcaattatttagcatttacttctgttcaaaataagtttgactctaccagtgtggatttaaaaaaatgaaatattaaataatattacattaaatataatttgcataccaaaatttatgcattttctgttgattcattcctgaggttggtgtgtctttctgaataaataagatagcaatagatttctctccaggatagtcattcttttctttattactcgagaaattttgatgttgtatacttaatgattggtggttcttctttgtttggccatcatgaaaattgccttctggtggcctatttttaagtggttagtgtttcactctaccaacttttacttcattattccaacaatatctagtttgttggtctcttttgtttttagactacacaaaaatactgaagattcaataggcaggtaacaaacattggctttctgtattatgttgttgagcatcacagtggtttcatagaaggacatggaaggaagcacagactttaaaagacacttgaggatatatatcactgaactattaggctgctattagcagaaatcaccagtgacatgcttattagactaatgtattttcattttaatactacatacatacttaacgaacctacaaattttttttgagcacctacctatgccagacactgtcaattgcttaatataaattaactgctttaaatctcattaacataatggagtctatgtggtaccccgattatactcatgaggaaactgaggcacagagaggttgttatttgcccaaggtatactaggaatagggccaggttacaaccccagatacctgggagtatacatttgagagagctcttggaggtaaaataaacagaacttggggactgagtgtagagtcttgagggaggaggagttaaagtccagtaagatttctactgctagacagggaaacatgaactggaagtgctccaagtaatagcaaccatgtgagcagaagcacaaaggtggaaaagtactctgggagccacagttagttcatatgatacatgaaataatgtaatgtcactttataggcaattggggcctcaaggggcttgaaaatgttggactagagtggtaatgcactgatactttctcagcagggagtagtatgatcacatctgctctaagagtgacacagttaaatagccctaggttttaaggtttactggcactatagtaaactattttttttaaatatttgattagttggatttttctgctttcaaccaggtggtttggtattatgtgagtagttcaattgtttattatttaaaaaaatactctgtaaaaccagagagaaccttggtaatattctgtatagaatccttcaaaggagtttctttattgctcagaaaatgtatttgatgggacagttgatcctcaaactcagtgactgttttgtagagcttaaagtaaattgaattacttgagttagaattatgtaattggaaagaaatgatataatatggaaacatacaaaatcttggaatatcattaaattatttttcaaaacatt

SEQ ID NO: 6 - DNA, 2317 bp, Homo sapiens
aaatctgtccgcccccttggccactgatcccccgaagagcttctgtcgccgctctaggaatagagacattgaagtttgggacaagatatttatctaacttctgtgtcaaaattagcgacctgctatggcaatgaagaaagaaactgaatttgtcattttcacctgaagaaaaatgatagacaaaaatcaaacctgtggtgtaggacaggattctgtgccctatatgatttgtctgattcacatactcgaagaatggtttggtgtggagcagttggaggactatttgaattttgcaaactatctcttgtgggtttttacaccactaatacttttaatacttccttactttactatctttcttctctaccttactattattttcttacacatttataagagaaagaatgtattgaaagaagcctactctcataatttatgggatggtgcaaggaaaacagtggcaactctgtgggatggacatgcagccgtttggcatggttatgaagttcatggaatggaaaaaataccagaagatggaccagcacttataattttttatcatggagctattcctatagatttttactatttcatggctaaaatatttatacacaaaggcagaacttgccgagtagtagctgatcactttgtctttaaaattccagggtttagtttattactggatgtgttttgtgctctacatggaccaagagaaaaatgtgttgaaattctgaggagtggccacttgttagctatctcaccaggtggagttcgagaagccctaattagtgatgaaacttataacatcgtatggggtcatcgcagaggctttgctcaggttgcaattgatgcaaaagtgacgaagaatgctgttcaagctttgattgataagcaccaaagaataccaggaaacattatgagtgctttgttagaacgttttcattgataacaaagggtcaactagaagatgatttagtacatttatattaaatgtttgtatctaaggtactgtcttctgaattttgtaggtcctataattagtattttttaaaaaaatcatgttaataagcatctttcacagaattcgtttctttaaaatagtcaattttgtttttgcaattgtgtcaaatactaacaaattacacacctagtaattcagaaaaagatgtcttatttgtaaattcctaacaatttatgctaaacatatagattcttaagtttattaataacagcagtttaggttaaacaaacattcctggataatgcgttaaatttctgtatctgtcgccctgagctgattttgaaagatggtataagctaggggttagtatagttgtttaagttagaaaaaacatgctgttgtctgcccctcattcccttcatgaccttgggcaagtcacgtaatgtttttgtgcctcaacaattcactttttaaaaacatgatcgtatgatgaatgatattattttgttatttatatttactgtgattgataactgttgaaccaaaataataaaataattaatttaaacaatgtcaaaatcctttagcagttatgtatatattttctccattgtgtgtttaaattatgtcatgtccagttgccaagcacaatgaaaaagatgtattattttttaaattgaataaaaaattaggaaaaataaaatttctaattattatttttagtatgatatttttaacaagagtctataggcaaacaatatagggtgtgctgtgcattgtcagccctatactgtggtcttaataatgccagcttaaaaatcactgttgtgctctgcatttcgtgtgttagaagctgattctaggctgaggaaagcaagagttctctacttttgctcaatattgaggcttacccagtttgactctacagccagtgaagtggtttattgcttcaataaaaatatacttgaatgatgaatttatttatgttttgttttgtttttatttagagatggggttttgccatgttggccaggctggtcttgaactcctgacctcaggtgatccacctgcctcagcctcccaacttgctgggattacaggtgtgagccaccatgcccagcctgaatgatgaatttatgtgaacacaaagccctctgaaaaattaatgattttcaatttgttgtcttcttgaaaactgaatttaagtattatttaaacagaaaagtgtcagaaatcaaatgcagtatctccctctcccttttttaaatgttgaattagtagggtttttgtatagctttttgtattgtgtgtatttatgatatgtgaaatgtacataaaattaaacaatgtgaaaat

SEQ ID NO: 7 - DNA, 3544 bp, Homo sapiens
tttttgaatctgcggaggcggcggcggtggcagcggcggcgcggcgactgaagcgcgcgaaaagctgaggcggcaacgtcggggacggctgcgcgggacggctctgtaggaaggaacttggttccccctccctcagcttccgccccaaaagattcagaatggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaaggtacttcaatagtaattgtactaagcaacgttttgataccattttttttgtcttaatacggtacagtattctacaaagacagatgtttatggcattatgcttggtttatttgtgttctttttggaatggaagttttttagattagaattttaaaatttggccaggtgcagtgtctcacacctgtaatcccagtgctttgggaggccaaggcaggaggatcgaggcaagaggtcaggagtttgagaccagcccgggcaatgcggtgaaaccccgtctctataaaaaatttaaaaattagctgggtgtgatggcatgtgtctgtggtcccagctgcatgggaggctgaggcaggaggatagcttgagcctgggaggttgaggctgccgtgagctgtgtttatgccactgcattccatcctgggtgacagaatgaggccctgtctcaa

SEQ ID NO: 8 - DNA, 4661 bp, Homo sapiens
tttttgaatctgcggaggcggcggcggtggcagcggcggcgcggcgactgaagcgcgcgaaaagctgaggcggcaacgtcggggacggctgcgcgggacggctctgtaggaaggaacttggttccccctccctcagcttccgccccaaaagattcagaatggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaagaccccaaaaaactaaatcacactcctgtatcaaccatgagttcttctcagcctgtgtcacgaccattgcaacccatacaaccagcaccgcctcttcaaccatctggggtgccaacaagtggaccatctcagaccaccatacacttactacctacagctccaactaccgtgaatgtaacacatcgtccagtaactcaggtgaccacaagactccctgtaccaagagctcctgcaaaccaccaggtggtttatacaactcttcctgcaccaccagctcaggctcccttgcgaggaactgttatgcaggctcctgctgttcggcaggtcaatccccaaaatagtgttacagttcgagtgcctcaaacaaccacatatgttgtaaacaatggactaaccctgggatcaacaggacctcagctcacagtgcatcaccgaccaccacaagtgcatactgagcccccacgccccgtgcacccagcacccttaccagaagctccacaaccacagcgtctgcccccagaagctgccagcacatctctgcctcagaagccacacttgaagttagcacgcgttcagagtcaaaatggcatagtactgtcatggagtgtcctggaggtggatcgaagctgtgccactgttgatagctaccatctctatgcttaccatgaggaacccagtgccactgtgccctcacaatggaaaaagattggggaagtcaaggcacttcccttgcccatggcatgtactctcacccagtttgtatctggtagcaaatactactttgcagtacgagccaaggatatttatggacgttttgggcctttctgtgatcctcagtcaacagatgtgatctcttctacccagagcagttaaaccttggagcctttatattttcctcttttaaaatttccaccttttggtcttgtttttaatcttgtgcatgataccccatgtaaaatccaccttgtgcaagatttcttggacagatgtgtgtatacactacatttgtttataaccagaagcaaaataaactcagcccacaaagctagaatcttttcctggacagtttaggctttggggtttggaaatgtaaatgtgtaccttgctttagttttgaggctggggaatatgtgtgggtgtttatgtgtgtttttccttatgtaggtgttattgcattggagtctcccattttcattctcaaatttacctcttaaagtacgaagtaagtagatcaaaggatttgagatgtgtaactggcatgattctgcttttgaaggatctatagtatcattttagttaagtgggtcaaacagaatcaaaacaaaacccaaagaaataaataaaaaacaaaatggctaaatagtttaaaataggttaattcgaacacaggaaaggatctatttgttgtttcttttgtctggtctcctgagttgttaattaggtgaaaaaagatctgcaatggccccctccctttcctaatctggcttttacatttattttgtgccttaaagattaactacaaagataaacatggccaaaaataaataaataaatatggccata

SEQ ID NO: 9 - DNA, 2711 bp, Homo sapiens
ggtgctgcggcccgcgccgccatcttggattttactctccatttttctctggaattatttttggtgattaattttctgggggggactgggacgcggggcccggcggcgcggccccgcatcgcagcggccgggcagcggggcctgggacgcgccccgaggaggagcggggcggcgcaggcggagagaacattgaaagtattctctaagctatttgaagagagtgactaaatgcacctgggtcaggctgtctgtgggtatgaagtggttgggagaatccaagaacatggtggtgaatggcaggagaaatggaggcaagttgtctaatgaccatcagcagaatcaatcaaaattacagcacacggggaaggacaccctgaaggctggcaaaaatgcagtcgagaggaggtcgaacagatgtaatggtaactcgggatttgaaggacagagtcgctatgtaccatcctctggaatgtccgccaaggaactctgtgaaaatgatgacctagcaaccagtttggttcttgatccctatttaggttttcaaacacacaaaatgaatactagcgcctttccttcgaggagctcaaggcatttttcaaaatctgacagtttttctcacaacaaccctgtgagatttaggcctattaaaggaaggcaggaagaactaaaggaagtaattgaacgttttaagaaagatgaacacttggagaaagccttcaaatgtttgacttcaggcgaatgggcacggcactattttctcaacaagaataaaatgcaggagaaattattcaaagaacatgtatttatttatttgcgaatgtttgcaactgacagtggatttgaaatattgccatgtaatagatactcatcagaacaaaatggagccaaaatagttgcaacaaaagagtggaaacgaaatgacaaaatagaattactggtgggttgtattgccgaactttcagaaattgaggagaacatgctacttagacatggagaaaacgacttcagtgtcatgtactccacaaggaaaaactgtgctcaactctggctgggtcctgctgcgtttataaaccatgattgcagacctaattgtaagtttgtgtcaactggtcgagatacagcatgtgtgaaggctctaagagacattgaacctggagaagaaatttcttgttattatggagatgggttctttggagaaaataatgagttctgcgagtgttacacttgcgaaagacggggcactggtgcttttaaatccagagtgggactgcctgcgcctgctcctgttatcaatagcaaatatggactcagagaaacagataaacgtttaaataggcttaaaaagttaggtgacagcagcaaaaattcagacagtcaatctgtcagctctaacactgatgcagataccactcaggaaaaaaacaatgcaagtaagtaagggagatttgataagcatatcttttaaaagtattttcacacaatttgctttataaagtgtgcttcagtagttttaaacttttaaatactcagagagactgggacttgtgagctttggctgcacttcaaggctctagacgtgatttgagtagaggcacagtctgtatcccatctctaacttcagtaccgtcctctagactatttttcttgaataccttggtaactggatatgagttcttcatcatatgttccaaggtcatcatatgttttaaacattttcaaggtgttagagactgtgatgatgtcgctaagtcctgcaagaagacaaaaggactgagtagaattaaattagactctatacattccagtgcctagccagtttgttagaaaagatgatggacttggggaattcatagcttctggccttaaggcttccaccttttcattgcttgctgacctttttcaaaacgaactgactcagttcagcagaccaccagtaccagactcagaattgtgatagaggagcattttgaacagtgccgtattgtgacatgctgtattggctactccagaaagtaggagtaaagatggaaaggagaaagaagcaacctctgagattccagtggtgtgtgggggcaagatctgatggaaactgaaaaagagaacgaagactaaacaaagagaaaggaaagagaagaaaccctaaatgggcaaaggaaagcacatcctgtttgcggagctttgaaatattggaaccatttctaattgctcctgtttttctgggtaacaccagttttctgtagttgccactaaagcagtagactcttgagtctcacttgtctctgagagagacagaagttagaaagttttgacttggcgattccgaaagtatgcctttgttggcacttaaatgtccagtgagacttcttggcaccttagagccctctgagatactgattattttaggttcttctccctactttcagatgttttcagcccaacactgggtgctctcttccactacagagaatcctgaagaaaagggaaggtgtttcccatgatggtgaatgtcactgccatgaattcctgaatctacctgctgctgggagtcagagtccaagcataacccgtgtagcataaaagcagcgctgtagccctattccagtctttttcgttaatgtccagagtgaacaacaagagttagtcaatcattaactgttgactgttgattctcataataaatgcagcataacgacaaaaaaaaaaaaaaaaaa

SEQ ID NO: 10 - DNA, 4562 bp, Homo sapiens
ggtgctgcggcccgcgccgccatcttggattttactctccatttttctctggaattatttttggtgattaattttctgggggggactgggacgcggggcccggcggcgcggccccgcatcgcagcggccgggcagcggggcctgggacgcgccccgaggaggagcggggcggcgcaggcggagagaacattgaaagtattctctaagctatttgaagagagtgactaaatgcacctgggtcaggctgtctgtgggtatgaagtggttgggagaatccaagaacatggtggtgaatggcaggagaaatggaggcaagttgtctaatgaccatcagcagaatcaatcaaaattacagcacacggggaaggacaccctgaaggctggcaaaaatgcagtcgagaggaggtcgaacagatgtaatggtaactcgggatttgaaggacagagtcgctatgtaccatcctctggaatgtccgccaaggaactctgtgaaaatgatgacctagcaaccagtttggttcttgatccctatttaggttttcaaacacacaaaatgaatactagcgcctttccttcgaggagctcaaggcatttttcaaaatctgacagtttttctcacaacaaccctgtgagatttaggcctattaaaggaaggcaggaagaactaaaggaagtaattgaacgttttaagaaagatgaacacttggagaaagccttcaaatgtttgacttcaggcgaatgggcacggcactattttctcaacaagaataaaatgcaggagaaattattcaaagaacatgtatttatttatttgcgaatgtttgcaactgacagtggatttgaaatattgccatgtaatagatactcatcagaacaaaatggagccaaaatagttgcaacaaaagagtggaaacgaaatgacaaaatagaattactggtgggttgtattgccgaactttcagaaattgaggagaacatgctacttagacatggagaaaacgacttcagtgtcatgtactccacaaggaaaaactgtgctcaactctggctgggtcctgctgcgtttataaaccatgattgcagacctaattgtaagtttgtgtcaactggtcgagatacagcatgtgtgaaggctctaagagacattgaacctggagaagaaatttcttgttattatggagatgggttctttggagaaaataatgagttctgcgagtgttacacttgcgaaagacggggcactggtgcttttaaatccagagtgggactgcctgcgcctgctcctgttatcaatagcaaatatggactcagagaaacagataaacgtttaaataggcttaaaaagttaggtgacagcagcaaaaattcagacagtcaatctgtcagctctaacactgatgcagataccactcaggaaaaaaacaatgcaacttctaaccgaaaatcttcagttggcgtaaaaaagaatagcaagagcagaacgttaacgaggcaatctatgtcaagaattccagcttcttccaactctacctcatctaagctaactcatataaataattccagggtaccaaagaaactgaagaagcctgcaaagcctttactttcaaagataaaattgagaaatcattgcaagcggctggagcaaaagaatgcttcaagaaaactcgaaatgggaaacttagtactgaaagagcctaaagtagttctgtataaaaatttgcccattaaaaaagataaggagccagagggaccagcccaagccgcagttgccagcgggtgcttgactagacacgcggcgagagaacacagacagaatcctgtgagaggtgctcattcgcagggggagagctcgccctgcacctacataactcggcggtcagtgaggacaagaacaaatctgaaggaggcctctgacatcaagcttgaaccaaatacgttgaatggctataaaagcagtgtgacggaaccttgccccgacagtggtgaacagctgcagccagctcctgtgctgcaggaggaagaactggctcatgagactgcacaaaaaggggaggcaaagtgtcataagagtgacacaggcatgtccaaaaagaagtcacgacaaggaaaacttgtgaaacagtttgcaaaaatagaggaatctactccagtgcacgattctcctggaaaagacgacgcggtaccagatttgatgggtccccattctgaccagggtgagcacagtggcactgtgggcgtgcctgtgagctacacagactgtgctccttcacccgtcggttgttcagttgtgacatcagatagcttcaaaacaaaagacagctttagaactgcaaaaagtaaaaagaagaggcgaatcacaaggtatgatgcacagttaatcctagaaaataactctgggattcccaaattgactcttcgtaggcgtcatgatagcagcagcaaaacaaatgaccaagagaatgatggaatgaactcttccaaaataagcatcaagttaagcaaagaccatgacaacgataacaatctctatgtagcaaagcttaataatggatttaactcaggatcaggcagtagttctacaaaattaaaaatccagctaaaacgagatgaggaaaatagggggtcttatacagaggggcttcatgaaaatggggtgtgctgcagtgatcctctttctctcttggagtctcgaatggaggtggatgactatagtcagtatgaggaagaaagtacagatgattcctcctcttctgagggcgatgaagaggaggatgactatgatgatgactttgaagacgattttattcctcttcctccagctaagcgcttgaggttaatagttggaaaagactctatagatattgacatttcttcaaggagaagagaagatcagtctttaaggcttaatgcctaagctcttggtcttaacttgacctgggataactactttaaagaaataaaaaattccagtcaattattcctcaactgaaagtttagtggcagcacttctattgtcccttcacttatcagcatactattgtagaaagtgtacagcatactgactcaattcttaagtctgatttgtgcaaatttttatcgtactttttaaatagccttcttacgtgcaattctgagttagaggtaaagccctgttgtaaaataaaggctcaagcaaaattgtacagtgatagcaactttccacacaggacgttgaaaacagtaatgtggctacacagtttttttaactgtaagagcatcagctggctctttaatatatgactaaacaataatttaaaacaaatcatagtagcagcatattaagggtttctagtatgctaatatcaccagcaatgatctttggctttttgatttatttgctagatgtttcccccttggagttttgtcagtttcacactgtttgctggcccaggtgtactgtttgtggcctttgttaatatcgcaaaccattggttgggagtcagattggtttcttaaaaaaaaaaaaaaaatgacatacgtgacagctcacttttcagttcattatatgtacgagggtagcagtgtgtgggatgaggttcgatacagcgtatttattgcttgtcatgtaaattaaaaaccttgtatttaactcttttcaatccttttagataaaattgttctttgcaagaatgattggtgcttattttttcaaaaatttgctgtgaacaacgtgatgacaacaagcaacatttatctaatgaactacagctatcttaatttggttcttcaagttttctgttgcacttgtaaaatgctacaaggaatattaaaaaaatctattcactttaacttataatagtttatgaaataaaaacatgagtcacagcttttgttctgtggtaacctataaaaaaagtttgtctttgagattcaatgtaaagaactgaaaacaatgtatatgttgtaaatatttgtgtgttgtgagaaatttttgtcataagaaattaaaagaacttaccaggaaggtttttaagtttagaaatattcatgccaataaaataggaaattataaatatatagttttaagcactgcatcagtgggagttcttggcttatgttagtttatgttagtttattatgaaaacatcaaagatttttttgactatattatcagttaaacaaaaaggagtcagatttaatttgttttttgaagcactttgagaaattaattttaattaacttaatgagcaaatttttattactactttatgttcaataccaggttcttttcatttctctggattattttgcaaatcattggacagagaatttgggaatataaatctgtaacaggtgtttgacaccagtaggtctctttatttctgggaaatgtgtacctgtactttctgatatacagtgttcctaagtaaaaatcaattcaggggatttgtatagtgtctataggaaagtagcccatgtcttgaaatatgaaaaggaatctgaaggtcatgaaaagtccagtggagaaaatctcaatgcttactgttactactaattgattcctactagtttccaggtttggg

SEQ ID NO: 11 - DNA, 2636 bp, Homo sapiens
gcggggagggggttgcaaaaggagccgagcggcttctgctcaatggcggaaaagccgccggtgctctgacggcctcgttcccctagcagttgcgggggagtttcctgccggcgcggctggagtctctgattctcagggttcggtggttggaagatgctccagagagacgaggctgcggcggaggaggtggcggcggccgaatcggcaacggcgctagggtggagagaaggcggcatcggcggcggcggcggcgtgaggggccgggcggtgtaaacagccccggaggcggcggtcgagaccccgagggggaagcggcggctgagtcagggtcgcgcctccgttggaaacttgggctgagtaccgcggcgggcgcgagcgaggcgccctagacatcttctccctcccttgcctcagatttattgctaaacatgggtgcatttttggataaacccaaaactgaaaaacataatgctcatggtgctgggaatggtttacgttatggcctgagcagcatgcaaggatggagagtggaaatggaagatgcacacacagctgttgtaggtattcctcacggcttggaagactggtcattttttgcagtttatgatggtcatgctggatcccgagtggcaaattactgctcaacacatttattagaacacatcactactaacgaagactttagggcagctggaaaatcaggatctgctcttgagctttcagtggaaaatgttaagaatggtatcagaactggatttttgaaaattgatgaatacatgcgtaacttttcagacctcagaaacgggatggacaggagtggttcaactgcagtgggagttatgatttcacctaagcatatctactttatcaactgtggtgattcacgtgctgttctgtataggaatggacaagtctgcttttctacccaggatcacaaaccttgcaatccaagggaaaaggagcgaatccaaaatgcaggaggcagcgtgatgatacaacgtgttaatggttcattagcagtatctcgtgctctgggggactatgattacaagtgtgttgatggcaagggcccaacagaacaacttgtttctccagagcctgaggtttatgaaattttaagagcagaagaggatgaatttatcatcttggcttgtgatgggatctgggatgttatgagtaatgaggagctctgtgaatatgttaaatctaggcttgaggtatctgatgacctggaaaatgtgtgcaattgggtagtggacacttgtttacacaagggaagtcgagataacatgagtattgtactagtttgcttttcaaatgctcccaaggtctcagatgaagcggtgaaaaaagattcagagttggataagcacttggaatcacgggttgaagagattatggagaagtctggcgaggaaggaatgcctgatcttgcccatgtcatgcgcatcttgtctgcagaaaatatcccaaatttgcctcctgggggaggtcttgctggcaagcgtaatgttattgaagctgtttatagtagactgaatccacatagagaaagtgatggggcctccgatgaagcagaggaaagtggatcacagggaaaattggtggaagctctcaggcaaatgagaattaatcataggggaaactaccgacaacttctggaggagatgctgactagttacaggctagctaaagtagagggagaagaaagccctgctgaaccagctgccacagctacttcttcgaacagtgatgctggaaacccagtgacaatgcaggaaagccatactgaatcagaaagtggtcttgctgaattagacagctctaatgaagatgcagggacaaagatgagtggtgaaaaaatatgactttcctttttggtaatatttttgtgatctttgatggtttttaacctaggaagtgtaatgtatgcatttatataactgttttgttatttgaatcttggaaaactagttttattatattcagatagccttgttttttaaaaaggcctttgcatacacctttatgagatagtgtaaaattgactatttatagtactatggatttaatgaaattatatgtcatttcacattgtatgccagaaattaggctaccaattatgaattaaagtcagtagttaaattaatactagatagaattagaaattttgattagagagattatgctatattatggaaaaacttgttaatgtagaattatactgcttcatattattttacctattagtacactcatagttagctttgtaataaatttatgttttctttaataattttagttcttcaaagaatggctgatgctggcctgtaatttttctttcaaggatgataatttgtgtgttgtttgatttgtttatattttacatctctgtagttttatttttagaagttgtgagatattggatgtgtggctatttttcctttctctgtattctttatgaaacataacttttgaaaaacctatgtattattcatacagctttggtttgtatattctgtatagcctaactacacacatcaaaatgtatgtcaaccaagtgtttagaatgaaattataagtgtttaagtccaaataaagcatgtgatgtggaataatctaaaaaaaaaaaaaaa

SEQ ID NO: 12 - DNA, 3850 bp, Homo sapiens
gcggggagggggttgcaaaaggagccgagcggcttctgctcaatggcggaaaagccgccggtgctctgacggcctcgttcccctagcagttgcgggggagtttcctgccggcgcggctggagtctctgattctcagggttcggtggttggaagatgctccagagagacgaggctgcggcggaggaggtggcggcggccgaatcggcaacggcgctagggtggagagaaggcggcatcggcggcggcggcggcgtgaggggccgggcggtgtaaacagccccggaggcggcggtcgagaccccgagggggaagcggcggctgagtcagggtcgcgcctccgttggaaacttgggctgagtaccgcggcgggcgcgagcgaggcgccctagacatcttctccctcccttgcctcagatttattgctaaacatgggtgcatttttggataaacccaaaactgaaaaacataatgctcatggtgctgggaatggtttacgttatggcctgagcagcatgcaaggatggagagtggaaatggaagatgcacacacagctgttgtaggtattcctcacggcttggaagactggtcattttttgcagtttatgatggtcatgctggatcccgagtggcaaattactgctcaacacatttattagaacacatcactactaacgaagactttagggcagctggaaaatcaggatctgctcttgagctttcagtggaaaatgttaagaatggtatcagaactggatttttgaaaattgatgaatacatgcgtaacttttcagacctcagaaacgggatggacaggagtggttcaactgcagtgggagttatgatttcacctaagcatatctactttatcaactgtggtgattcacgtgctgttctgtataggaatggacaagtctgcttttctacccaggatcacaaaccttgcaatccaagggaaaaggagcgaatccaaaatgcaggaggcagcgtgatgatacaacgtgttaatggttcattagcagtatctcgtgctctgggggactatgattacaagtgtgttgatggcaagggcccaacagaacaacttgtttctccagagcctgaggtttatgaaattttaagagcagaagaggatgaatttatcatcttggcttgtgatgggatctgggatgttatgagtaatgaggagctctgtgaatatgttaaatctaggcttgaggtatctgatgacctggaaaatgtgtgcaattgggtagtggacacttgtttacacaagggaagtcgagataacatgagtattgtactagtttgcttttcaaatgctcccaaggtctcagatgaagcggtgaaaaaagattcagagttggataagcacttggaatcacgggttgaagagattatggagaagtctggcgaggaaggaatgcctgatcttgcccatgtcatgcgcatcttgtctgcagaaaatatcccaaatttgcctcctgggggaggtcttgctggcaagcgtaatgttattgaagctgtttatagtagactgaatccacatagagaaagtgatgggggtgctggagatctagaagacccatggtagccttaaaaaccttctaaaatgcttttgattctgaaaattgggggaaaaaacttttaatcacaattttcttcaatacaaggggaaaatattcttgcggattcccaacgttttgtgatatgagcagaaaatcattagcatttcccatcatttgttcatatttgtgttttctgacagttgccacttgtagcattgcctgtactacagtattttttgccaacctcaggcatactcgttacatctgtattgaactttcggccctagaaaccagtggagttatttcaccacaaatcaacaatgtgcctgaggtgcatgggaaatatagttagctatactctgaaaatacattatgttttttttctttaaacaaaacacacaacatgtaagcatgtaagagtaaagaattgtatgatatgttccttttttcagttcaccaagttggaagccttttgcagctctgtggcttggaatttcatttgagcaatttctataggatatgtatttattattgattgttatttaatttttttccaattttacctgtattaccaaactgggttctccaataatgtccaaattgtaatgttgccttgcttcaagataaagtgtatttgggaataatattataaacccttacaaattttatgcatgtatctactgcatccttcaactctcactagaaaatcttttgaaaccaaatggattaatttatggctatttataatttgctttgacatctcactgttggaaattttttaaagatgagatttgcctttataatgtaaattgtgatttttgttttacatgtgggtttctatagttttaattttttcagcttttaagatacgagttttgtgtaatttggtatttttaatcatttatgttattttaaaagctcagaatatcacattgaaattactataaatacatttaaaattatctattttagatctaaggaaatactacagagatattttcatgggttcagtaacttttcattttataacattgggcacggtacagagtgattgtcacataaggtacttgaagatttattagtttaattctatttttacagtaaccttgaattcttctgagttttgcatgtattaaattcaattaatgctgaacatgaagagtaaagtatttatctgaaagaagtttctgggttaggagaagtaatgaatgtatccatttgtacatggtttacatgttgtggatgctttgtaaacattttcctgtatgtttaaattgtgtttcagcaggatgtaattgcccttgtgtgtagttaaaatgagtcatcatctggtcctttgtgaaatggaattcatggtattttctgtaacgttttcctgaagctgtttctggagagccacacatttaaatacagacagctttcctgatcatttgatttattgtgcacctgatttttggtctaaaaggaattattgccacaatatattttatttattctttagattttagccttgtaagttaaagtgctttacatgatgatgtgaaaagctgtttgtccctttactgggtttggggggttgttaaaagatagggaatgaagaatgcaaaatggtttatcgttcaaactgtccactctgatccaaccctgtactgatagtacttcccagtatgatattgtgatgtttcatacaatgcagtgaacataaccaacttgttacctaaataaagaattgataaaaacagtgtgacatattactatttggtatgattcagtttttaatgttattttaaacttcttatatggttggcatatttctgaaaatttaattccaaagaggaattaaccaaactgtacaggacaatattaaccccttctgtactctagaaatatttatactccttttaaaaaaacctgcctttggccgggtgcattggctcacacctgtaatcccagcactttgggaggccaaggcgggtggatcacgaggtcaagagatcgtgaccatcctggccaacacggtgaaaccctgcctctactaaaaatacaaagattagctgggcatggtggtgcttgcctgtaatcccagctgcttgggaggctgaggcagaagaattgcttgaacccaggaggcggaggttgcagtgagctaagatcgcgccactgcactccagcctggcgacagagcgagactccgtctcaaaaaaaaaaaaaaagaaaaaaaaaaacttttccggcagggggcagggtgggggttactggtgcttgaaacagaaataatgtatga

SEQ ID NO: 13 - DNA, 6135 bp, Homo sapiens
tgcctcgtccgccggctacgtctgcgtttccgcctttccttttgtttttctcaggttttgcgtgggaggcggtcccgggatttcaagggtctacgcgcttttctatggcgaatgcaacccgacgagggagtgggctgtatcttcagagttgtctccgtctttccaagaacagaacaaaatgaacaaggtagaacagaagtcccaggagtcagtatcatttaaagatgtgactgtgggcttcacccaggaggagtggcagcacctggaccctagtcagagggctctgtatagagatgtgatgctggagaactacagcaaccttgtctcagtgggtaagggtattgtgttcacaaaccagaggtgatcttcaggctgcaacaaggagaagagccatggaaacaggaggaagaattcccaagccaaagctttccagaagtctggacagctgatcacctgaaagagaggagccaagaaaaccaatctaaacatttgtgggaagttgtattcatcaataatgaaatgctgactaaggaacaaggtgatgtaataggaataccatttaatgtggatgtaagttcttttccttccagaaaaatgttctgtcagtgtgattcatgtggaatgagtttcaacactgtttcagaattggttatcagtaagataaactatttaggaaaaaagtctgatgaatttaatgcctgtgggaaattgttactcaatattaagcatgatgaaactcatactcaagagaaaaatgaagttttgaaaaataggaacacactgagtcatcatgaggagactttgcagcatgagaagattcaaactttagagcacaattttgaatacagtatatgtcaggaaaccctccttgaaaaggcagtattcaatacacagaagagagagaacgcagaagagaataactgtgattataatgaatttgggagaactttgtgtgatagttcatccctcttgttccatcagatatctccgtcaagggacaatcactatgaatttagtgattgtgagaagttcttatgtgtgaagtccaccctttctaaacctcatggggtatctatgaaacactatgattgtggtgaaagtgggaataatttcaggaggaaattgtgtctgtcacaccttcagaaaggtgataaaggagagaaacactttgaatgtaatgaatgtgggaaagctttctgggagaagtcacatctcactcgacatcagagggtgcacacaggacagaaaccctttcaatgtaatgaatgtgaaaaagctttctgggataagtcaaacctcactaaacatcaaagatcacacacaggggagaaaccttttgaatgcaatgaatgtgggaaagcctttagccataagtcagccctcacattacaccagagaacacatacaggggagaaaccctatcaatgtaatgcgtgtgggaaaactttttgccagaaatctgacctcactaaacatcagagaacacacacagggctgaaaccctatgaatgttatgaatgtggaaaatccttccgtgtgacttcgcaccttaaagtacaccagagaactcacacaggtgagaaaccttttgaatgtcttgagtgtgggaaatcctttagtgaaaagtcaaatcttacacagcatcagagaattcacataggagataaatcttatgaatgtaatgcatgtgggaaaactttctaccacaagtcattactcaccaggcatcagataattcatacagggtggaaaccttatgaatgttatgaatgtgggaaaaccttctgcttgaagtcagacctcacagtacatcagagaacacacacagggcagaaaccctttgcatgtcccgaatgtgggaaattctttagccataagtcaaccctctctcaacattatagaacacacacaggggagaaaccctacgaatgtcatgaatgtggaaaaatcttttacaataaatcatacctaactaaacataatagaacacatacaggggagaaaccctatgaatgtaatgaatgtggaaaagccttctaccagaagtcacaactcactcagcatcagagaattcacataggggagaaaccctataaatgtaatgagtgtggaaaagctttctgccataagtcagctctaattgtacatcagagaacccatacacaagaaaagccctataaatgtaatgaatgtggaaaatctttctgtgtaaaatcaggacttattttccatgagagaaagcacacgggggagaaaccctatgaatgcaatgaatgtgggaaattcttcaggcacaaatcatcactcacagtacatcacagggctcacacaggagagaaatcttgtcaatgtaatgaatgtggaaaaatcttttaccgtaaatcggaacttgctcaacatcagagatcacatacaggggaaaagccctatgaatgtaacacatgcaggaaaactttctctcaaaagtcaaatctcattgtacatcagagaagacatataggagaaaaccttatgaatgaaatggatattagaaatttccagccacaagtcagcctccataatgcctcagagtattcacactgtggagaaagccctgatgacatcctgaatgttcagtaactatccacaaactcaccttatgttactccaaagtaatagtaggggataaacccatagactacaacaattataggacagcttttgttaggaagtgatattctatgtaatatcagatggttaatacgggcataacaccttacagattttttttgaatttgtgaaagtttttggcaaaaatgcaaataaggttatgttagaatttacactgaggagaaaattgtcaatttaagaaatctaaagtgaaaattttgcttagaaataaaatacgacaagttatgttttgagtttgatgccataatagtttttagggcacctaacaataatttatagatgtatattgtggaatgtaaagctttaagaacttaaacaaaggtaataattaaaataacctcacaagaagtttaatgagtagagagaattcccatgtacacttcaccaaacttcctctaaggataatgtataatcatagtatatggtcaagtccaagaaatcgatgttaccttgctggtagatagagacttagtcagattttactatggtttgcatgcactctgtgtgtgtgtgtacgtgtatgtgtgtgtgtgtggttatatcaaacttttacgacttttacatttgagtaaccatcaacacgattagtttacagaactgaaaaggaatctgagatctgtactgtatcaggattacaaaggatttagcctcaagtagttccctttttgtattaataaccacacgctttctgcaaccctatcccttgcatccacttgactatgaagtttttttaaagcacagataaattgaataatcagggcaatagcattaagcacatctgcgaattatccctgaagttcaaaagacttatatatgtataagtggtatgtgatataaatcgtgtgtttcatatgcataatggaatttaacgtaattgtgaataggaagtaggtatcctagtttagtagtggttacattatcaggcccatcccagatgtttgtgatgtcctgaaacaatttaaaaggagacccacagagctaatgtttatccatttaaaaggtataaatcaaaataacaaattatctaaaaaaaatctatcctgtacatgtgaagatctaggctcgcctccatgttactccctttctcttcctacctgtggctcatgccatgccatgaagtgcctagggtacatatgtgaacattccagccttcagcataagtggtagtccaaattttctttaagcagctgctctccaagacagggccctggaaaggcccacacatacagcccagggctgcccagggctgttggactgtcatttcaaggctcattgtccccagacagggatctgggccctcagacttttgagaatatgaagaggagggttgaggctggagcagaaccagaatcaagctggtgtggggcttttattgatagtgtgaagattgtacactatattacatgatcagatcatacaaataatctaagattatacagtctagtgatcctagattacacaagtaatctaagcttccactttaggaaactagaaaaagaagagcaaattaaatccaaagtaacctgaaaaaacattagagtaaaaatcattgaaattgaaaagagaaaatcgataaaacccaaaactggttccttgaaaaggtcagtaaaattgataaacctctagctagcttaagaacaaacagaagacaaatattgctaataccaaaaatgaaagaggccatcacagctaatcctatggtcaccaaaaggattagaaagaaacatggacaactctgtccataaatttgataacttagctaaaatggaccaattccttgcaagacgcattctctcaaagctcacacaagatagacctcaataggcatatgtatctattaaataaaccaaagcagtaattaataaccttccaaggccaggtacagtggttcatgcctgtaatcccagcacttcactttgggaggctgaggcgggtggatcacaaggtcaggagttcgagaccagcctggtcaacatggtgaaaccccctatctactaaaaatacaaaaaattagctgggcatggtggcatgcgtgtgtagtcgaagctactcaggaggctgaggcagaagaatcacttgaacctgggaggtggatgttgcagtgagctgagattgtgccactgtactgcagcctgggtgacagaatgagactgcatctcaaaaaataataatgaaactttccaaaacaaagtgccaggaccagctgggttcactgatgtgtttttgagatacttaaggtagaaatgataccaagtctctaccatctctttcagaaaataaaagcagagataacacttcccaactcattttgtgaggccagtattaccctaatagcaaaaccaggcaaagatattacaagaaaggaaaactacagatcaatatttccattgaacacagatgcagaaatcctcaaaatattagcaaatcaggaatgcaaggcttgttcaacatttgatattcagtgtaatccattgcatgcattagcagctcaaagatgaaatacgatcatatcaataggtacagaaaaagcatttgacaaaatccaatatccattcatgttaaaacctctcaacacagaaggaatacaggagaacttactcaagttggtaaagaacatactacaaaaaaccctacagctaacattatccttgatgtttccaactagacactttcctactaagatcaggaacatggcaaggaacaccacaccactgcttttcagcattgtactggaagtgctatataatgcagtgttaaaaaaaaaagaaataaaatgcacatagatttgaaaggatgaaatacaactttgttcacaagtaacatgattgtctttgtagagaattctaaataacaaaaaccctgctgacattaataagcaattacagcaggattgcaggatacagtcctcaggtagatgataaaaaggaattggacatcaagtcattaaaagacacggagaaaccctaaatgcttattgttaagtgaaaaccaatctgaaaaggctttctattttgtatgattgcaattatatgacattttgggagaggcaaaactattgagccagtgaaaggaccagtggttgctgagagttgaggggaaaggagggatggcaggtagagaggtgatttttaaggcagtgaaagtactctctagggtactgtaatttgtcaaaacgcagagaatggaaaacagtgaacggaaaacacagagtaaaccctaatgtcaactctggacttcagttaataataatgtatcactgttggctcattaattgtaacaaatgaaccataatgatgcaagatgttaatgattggggagcctgtggaagatgggaggggaaagggttatatgagaattctctatactttttgctcaactttctgtaaccctaaagctactctaaaaaatgaagtgtatcaagtaaataaataaccaaccaaccaaccctgaggaacaaaaaaaaaa

SEQ ID NO: 14 - DNA, 1576 bp, Homo sapiens
tgcctcgtccgccggctacgtctgcgtttccgcctttccttttgtttttctcaggttttgcgtgggaggcggtcccgggatttcaagggtctacgcgcttttctatggcgaatgcaacccgacgagggagtgggctgtatcttcagagttgtctccgtctttccaagaacagaacaaaatgaacaaggtagaacagaagtcccaggagtcagtatcatttaaagatgtgactgtgggcttcacccaggaggagtggcagcacctggaccctagtcagagggctctgtatagagatgtgatgctggagaactacagcaaccttgtctcagtggggtattgtgttcacaaaccagaggtgatcttcaggctgcaacaaggagaagagccatggaaacaggaggaagaattcccaagccaaagctttccagaagtctggacagctgatcacctgaaagagaggagccaagaaaaccaatctaaacatttgtgggaagttgtattcatcaataatgaaatgctgactaaggaacaaggtgatgtaataggaataccatttaatgtggatgcccctgagatgctcatgcccttggattaaacccctgcagtgctggaagaagaggactctgcactgcctgccacaccaggactggaagatccatggccttcccttcttccaaactaccttcccacaagatggggatggtggtcacatctccaacactagcatgctgtggaagagacatcagcattcttgcaacaaatctggaaacagcctaaagagacatttctacttcaaagagttgcactgaggaagtgcactggcactgctggggactcacctggggcccagggctgaggcttctcgtgtccttggagctgctgtcctcccgaggagtgtccacactgcagccagctcagcctcaggtgtcttctctggttttgaaaccaaatctaagtgggaaaagaagattcaagttttatccgatttagtctacataattttaagtatctatttgagctcaataccaagaatcacttccttctctctcccatctgtgatctccagaggaaaggtccccgggctaggagacactggctggcatcctaaagccagagctggccaaagggtgtgtgtccactcttccttcacctcttccccaccccacacttgtttttgtctcacgctcacttgctctttctgcctccctctcactctttaggtctcacaaaggctttggccaaagactgactgactttccttggctctctgtcatctccccatagtcaaaagcatttcataacacacaatccatgtgaagggacctctgatgaaaatccatttaaagaggagctcactaattatcaatacttactgctgagcctggaatcattcatatatacctcagagttcattcattccagatgcaatactagataaggagactcctatgaaccaaatatctaactctgaatagatagtgactgtcataatcaaaccctgtggctgcctcctccttactggagggtgtagaggaatgcagttttttgtttgtttatttgtttgttttgagatcat

SEQ ID NO: 15 - DNA, 25 bp, Homo sapiens
gaatttatacaaggagaagcactgg

SEQ ID NO: 16 - DNA, 20 bp, Homo sapiens
actaagcagaagccttgacc

SEQ ID NO: 17 - DNA, 24 bp, Homo sapiens
caagccaaatactccactattgag

SEQ ID NO: 18 - DNA, 20 bp, Homo sapiens
gcattgctcagctactggag

SEQ ID NO: 19 - DNA, 22 bp, Homo sapiens
actacaaggatcacggaaactg

SEQ ID NO: 20 - DNA, 21 bp, Homo sapiens
aaagggtctcagaaacacagg

SEQ ID NO: 21 - DNA, 20 bp, Homo sapiens
cccttgtttgatgtcactgc

SEQ ID NO: 22 - DNA, 20 bp, Homo sapiens
ccaggaccatcccttcattc

SEQ ID NO: 23 - DNA, 20 bp, Homo sapiens
aactctgtgggatggacatg

SEQ ID NO: 24 - DNA, 21 bp, Homo sapiens
acatgaatggcaacacaaagg

SEQ ID NO: 25 - DNA, 21 bp, Homo sapiens
gatggtgcaaggaaaacagtg

SEQ ID NO: 26 - DNA, 26 bp, Homo sapiens
aaaattataagtgctggtccatcttc

SEQ ID NO: 27 - DNA, 21 bp, Homo sapiens
ctcacacctgtaatcccagtg

SEQ ID NO: 28 - DNA, 22 bp, Homo sapiens
gtctcaaactcctgacctcttg

SEQ ID NO: 29 - DNA, 19 bp, Homo sapiens
tcagctcacagtgcatcac

SEQ ID NO: 30 - DNA, 20 bp, Homo sapiens
ttcaagtgtggcttctgagg

SEQ ID NO: 31 - DNA, 20 bp, Homo sapiens
atgtcgctaagtcctgcaag

SEQ ID NO: 32 - DNA, 20 bp, Homo sapiens
gcaatgaaaaggtggaagcc

SEQ ID NO: 33 - DNA, 24 bp, Homo sapiens
tggactcagagaaacagataaacg

SEQ ID NO: 34 - DNA, 25 bp, Homo sapiens
actgaagattttcggttagaagttg

SEQ ID NO: 35 - DNA, 23 bp, Homo sapiens
atccacatagagaaagtgatggg

SEQ ID NO: 36 - DNA, 22 bp, Homo sapiens
gttgtcggtagtttcccctatg

SEQ ID NO: 37 - DNA, 23 bp, Homo sapiens
tggcaagcgtaatgttattgaag

SEQ ID NO: 38 - DNA, 23 bp, Homo sapiens
ccccaattttcagaatcaaaagc

SEQ ID NO: 39 - DNA, 20 bp, Homo sapiens
acagcaaccttgtctcagtg

SEQ ID NO: 40 - DNA, 20 bp, Homo sapiens
tcctgtttccatggctcttc

SEQ ID NO: 41 - DNA, 26 bp, Homo sapiens
ataggaataccatttaatgtggatgc

SEQ ID NO: 42 - DNA, 21 bp, Homo sapiens
tgtgggaaggtagtttggaag

SEQ ID NO: 43 - DNA, 24 bp, Homo sapiens
actgaaaatggctgggttgcagac

SEQ ID NO: 44 - DNA, 28 bp, Homo sapiens
agcctgaggttactgcattaaaagtgcc

SEQ ID NO: 45 - DNA, 27 bp, Homo sapiens
acgggaactgcaatacacaaaaactgg

SEQ ID NO: 46 - DNA, 24 bp, Homo sapiens
tctcctggtttctcgttgcccatt

SEQ ID NO: 47 - DNA, 24 bp, Homo sapiens
ccgtttggcatggtaagcaaggat

SEQ ID NO: 48 - DNA, 22 bp, Homo sapiens
caaacggctgcatgtccatccc

SEQ ID NO: 49 - DNA, 20 bp, Homo sapiens
atcctcctgccttggcctcc

SEQ ID NO: 50 - DNA, 23 bp, Homo sapiens
tgtggagcttctggtaagggtgc

SEQ ID NO: 51 - DNA, 27 bp, Homo sapiens
ccagaagctatgaattccccaagtcca

SEQ ID NO: 52 - DNA, 27 bp, Homo sapiens
ctgtctgaatttttgctgctgtcacct

SEQ ID NO: 53 - DNA, 26 bp, Homo sapiens
tttccctgtgatccactttcctctgc

SEQ ID NO: 54 - DNA, 24 bp, Homo sapiens
atctccagcacccccatcactttc

SEQ ID NO: 55 - DNA, 25 bp, Homo sapiens
cagaggtgatcttcaggctgcaaca

SEQ ID NO: 56 - DNA, 24 bp, Homo sapiens
cctgagatgctcatgcccttggat

(19) United States
(12) Patent Application Publication
(10) Pub. No.: US 2017/0283882 A1
(43) Pub. Date: Oct. 5, 2017

(54) METHODS AND THERAPEUTICS RELATING TO MRNA BIOMARKERS FOR CLINICAL PROGNOSIS OF CANCER

(71) Applicant: Duke University, Durham, NC (US)

(72) Inventors: Timothy J. Robinson, Durham, NC (US); Mark W. Dewhirst, Chapel Hill, NC (US)

(21) Appl. No.: 15/508,770

(22) PCT Filed: Sep. 8, 2015
(86) PCT No.: PCT/US15/48998

§ 371 Date: Mar. 3, 2017

Related U.S. Application Data
(60) Provisional application No. 62/046,221, filed on Sep. 5, 2014.

(51) Int. Cl. C12Q 1/68

(52) U.S. Cl. CPC C12Q 1/6886; C12Q 2600/118; C12Q 2600/158

(57) ABSTRACT

This disclosure provides methods and compositions of matter relating to the monitoring of relative concentrations of pairs of isoforms of mRNA or downstream expression products thereof. The methods can include measuring a relative expression level of a pair of mRNA isoforms or downstream expression products thereof and comparing the relative expression level to relative historical or cohort expression levels of the same pair of mRNA isoforms. The result of the comparison can lead to a more accurate prognosis for the subject.

### CROSS-REFERENCE TO RELATED APPLICATIONS

This application is based on, incorporates herein by reference, and claims the benefit of U.S. Provisional Patent Application No. 62/046,221, filed Sep. 5, 2014, and entitled “METHODS AND THERAPEUTICS RELATING TO MRNA BIOMARKERS FOR THE CLINICAL PROGNOSIS OF CANCER”.

### STATEMENT REGARDING FEDERALLY SPONSORED RESEARCH

This invention was made with government support under CA40355 entitled “Heat and Radiation Effects in Tumor Microcirculation” awarded by NIH/NCI. The government has certain rights in the invention.

### SEQUENCE LISTING

The sequence listing is filed with the application in electronic format only and is incorporated by reference herein. The sequence listing text file “DU4226PCT_ST25.txt” was created on Sep. 7, 2015 and is 73,228 bytes in size.

### BACKGROUND

### 1. Field of the Invention

This invention relates to methods and therapeutics relating to mRNA biomarkers for clinical prognosis of cancer.

### 2. Description of the Related Art

The role of varying mRNA isoform concentrations in clinical cancer prognosis has been limited.

Accordingly, a need exists for methods and therapeutics that can provide improved prognosis capabilities and corresponding outcomes in cancer patients.

### SUMMARY

In an aspect, the present disclosure provides a method of providing a prognosis to a subject suffering from an adenocarcinoma is provided. The method can include one or more of the following steps: obtaining a biological sample from the subject; measuring a relative expression level of one or more pairs of mRNA isoforms or downstream expression products thereof within the biological sample; and comparing the relative expression level to relative historical or contemporary cohort expression levels of the one or more pairs of mRNA isoforms or downstream expression products thereof, the relative historical or contemporary cohort expression levels retrieved from a historical or contemporary data set for historical or contemporary patients suffering from the adenocarcinoma.

In another aspects, the present disclosure provides a method of identifying predictive targets in a cancer of interest. The method can include one or more of the following steps: inducing an induced state in a plurality of inducement subjects having the cancer of interest; acquiring a plurality of induced biological samples by acquiring an induced biological sample from each of the plurality of inducement subjects in the induced state; acquiring a plurality of control biological samples by acquiring a control biological sample from each of a plurality of control subjects in a control state, the plurality of control subjects having the cancer of interest; measuring relative expression levels of one or more pairs of mRNA isoforms or downstream expression products thereof within the plurality of induced biological samples and the plurality of control biological samples and recording the relative expression levels to an expression data set; applying selection criteria to the expression data set to identify preliminary targets; validating the preliminary targets resulting in validated targets; and identifying statistically significant associations between the validated targets and an outcome of interest in a historical data set, the historical data set including historical relative expression levels of the one or more pairs of mRNA isoforms or downstream expression products thereof for a plurality of historical patients that had the cancer of interest. The induced state can be hypoxia. The control state can be normoxia.

In yet another aspects, this disclosure provides compositions of matter. The composition of matter can include an artificially synthesized polymerase chain reaction primer selected from the group consisting of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42. The composition of matter can include an artificially synthesized probe selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.

These and other features, aspects, and advantages of the present invention will become better understood upon consideration of the following detailed description, drawings and appended claims.

### BRIEF DESCRIPTION OF THE DRAWINGS

FIG. 1 is a flowchart showing a method in accordance with one aspect of the present disclosure.

FIG. 2 is a flowchart showing a method in accordance with one aspect of the present disclosure.

FIG. 3 is a plot showing the RT-qPCR validation data for ATF7IP of Example 2.

FIG. 4 is a plot showing the RT-qPCR validation data for PPM of Example 2.

FIG. 5 is a plot showing the RT-qPCR validation data for ZNF33A of Example 2.

FIG. 6 is a plot showing the RT-qPCR validation data for ETNK1 of Example 2.

FIG. 7 is a plot showing the RT-qPCR validation data for CARD8 of Example 2.

FIG. 8 is a plot showing the RT-qPCR validation data for TMEM68 of Example 2.

FIG. 9 is a plot showing the RT-qPCR validation data for SUV420H1 of Example 2.

FIG. 10 is a plot showing the RT-qPCR validation data for NUP160 of Example 2.

FIG. 11 is an image of a Western blot of ATF7IP of Example 2.

FIG. 12 is an image of a Western blot of PPM1B of Example 2.

FIG. 13 is a plot comparing survival probability for patients having a top 50th percentile AFT7IP isoform ratio and patients having a lower 50th percentile ATF7IP isoform ratio in the Lee cohort of Example 2.

FIG. 14 is a plot comparing survival probability for patients having a top 50th percentile AFT7IP isoform ratio and patients having a lower 50th percentile ATF7IP isoform ratio in the Okayama cohort of Example 2.

FIG. 15 is a plot comparing survival probability for patients having a top 80th percentile PPM1B isoform ratio and patients having a lower 20th percentile PPM1B isoform ratio in the Lee cohort of Example 2.

FIG. 16 is a plot comparing survival probability for patients having a top 80th percentile PPM1B isoform ratio and patients having a lower 20th percentile PPM1B isoform ratio in the Okayama cohort of Example 2.

FIG. 17 is a plot comparing survival probability for patients having a top 80th percentile ETNK1 isoform ratio and patients having a lower 20th percentile ETNK1 isoform ratio in the Lee cohort of Example 2.

FIG. 18 is a plot comparing survival probability for patients having a top 80th percentile ETNK1 isoform ratio and patients having a lower 20th percentile ETNK1 isoform ratio in the Okayama cohort of Example 2.

FIG. 19 is a plot comparing survival probability for patients having a top 80th percentile ETNK1 isoform ratio and patients having a lower 20th percentile ETNK1 isoform ratio in the Lee cohort of Example 2.

FIG. 20 is a plot comparing survival probability for patients having a top 80th percentile ETNK1 isoform ratio and patients having a lower 20th percentile ETNK1 isoform ratio in the Okayama cohort of Example 2.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Features of this disclosure described with respect to a particular method, apparatus, composition, or other aspect of the disclosure can be combined with, substituted for, integrated into, or in any other way utilized with other methods, apparatuses, compositions, or other aspects of the disclosure, unless explicitly indicated otherwise or necessitated by the context. For clarity, an aspect of the invention described with respect to one method can be utilized in other methods described herein, or in apparatuses or with compositions described herein.

### Definitions

As used herein, the term “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “biomarker” refers to a naturally occurring biological molecule present in a subject at varying concentrations that is useful in determining a given disease state. A biomarker can include relative expression levels of pairs of mRNA isoforms, as described herein.

The terms “over expression”, high levels”, “increased levels”, “high expression”, “increased expression” or “elevated levels” in regards to gene expression/mRNA levels are used herein interchangeably to refer to expression of a gene in a cell or population of cells, particularly a cancer cell or population of cancer cells, at levels higher than the expression of that gene in a second cell or population of cells, for example normal (i.e., noncancererous) cells or other control. “Elevated levels” of gene expression can refer to expression of a gene in a cancer cell or population of cancer cells at levels once that, twice that or more of expression levels of the same gene in normal/control cells. “Elevated levels” of gene expression can be determined by detecting increased amounts of a polynucleotide (mRNA, cDNA, etc.) in cancer cells compared to normal/control cells by, for example, quantitative RT-PCR or microarray analysis. Alternatively “elevated levels” of gene expression can be determined by detecting increased amounts of a protein in cancer cells compared to normal/control cells by, for example, ELISA, Western blot, quantitative immunofluorescence, etc.

As used herein, the terms “cancer” and “cancerous” refer to or describe the physiological condition in mammals in which a population of cells are characterized by unregulated cell growth. A cancer may be a non-solid tumor type or a solid tumor. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, the term “subject suffering from cancer” refers to a subject that presents one or more symptoms indicative of a cancer (e.g., a noticeable lump or mass) or has been diagnosed as having cancer.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. For example, such therapies may include surgery, medications (hormonal therapy and/or chemotherapy), radiation, immunotherapy and the like. Such treatments are well known and particular to the patient and can be readily determined by one skilled in the art.

The term “effective amount” or “therapeutically effective amount” refers to an amount sufficient to effect beneficial or desirable biological and/or clinical results.

The term “biological sample” refers to a sample containing tissues, cells, biological materials, and/or biological fluids isolated from a subject.

The term “hypoxia upregulated” refers to an mRNA isoform that is upregulated in a hypoxic state relative to a normoxic state. The term “hypoxia downregulated” refers to an mRNA isoform that is downregulated in a hypoxic state relative to a normoxic state.

### Methods of Providing a Prognosis to a Subject Suffering From an Adenocarcinoma

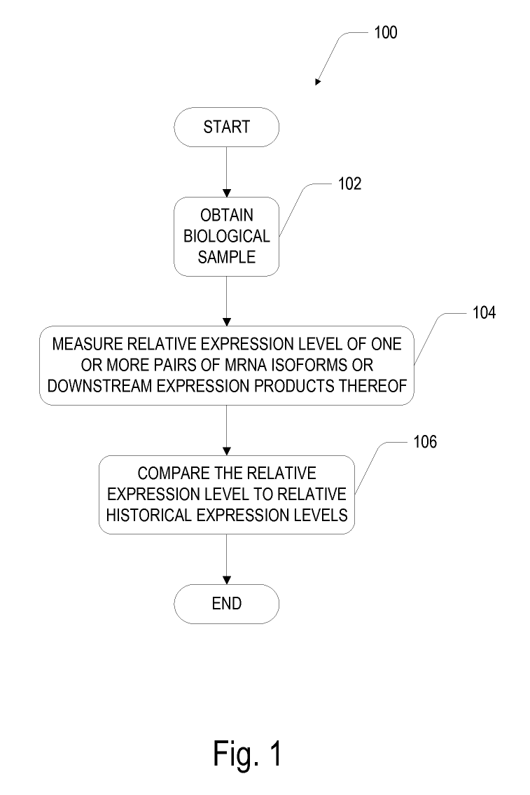

Referring to FIG. 1, this disclosure provides a method 100 of providing a prognosis to a subject suffering from an adenocarcinoma. At process block 102, the method 100 can include obtaining a biological sample from the subject. At process block 104, the method 100 can include measuring a relative expression level of one or more pairs of mRNA isoforms or downstream expression products thereof within the biological sample. At process block 106, the method can include comparing the relative expression level to relative historical or contemporary cohort expression levels of the one or more pairs of mRNA isoforms or downstream expression products thereof. The relative historical expression levels can be retrieved from a historical survival data set for historical patients suffering from the adenocarcinoma. The relative contemporary cohort expression levels can be retrieved from a contemporary data set for contemporary patients suffering from the adenocarcinoma.

In certain aspects, the method 100 can be a method of determining tumor hypoxia in a subject suffering from an adenocarcenoma, and the method 100 can include substantially the same steps as described above with respect to the aspects relating to a prognosis. It should be appreciated that a person having ordinary skill in the art could identify a prognosis or determine tumor hypoxia based on the outcome of process block 106.

### Methods of Identifying Predictive Targets in a Cancer of Interest

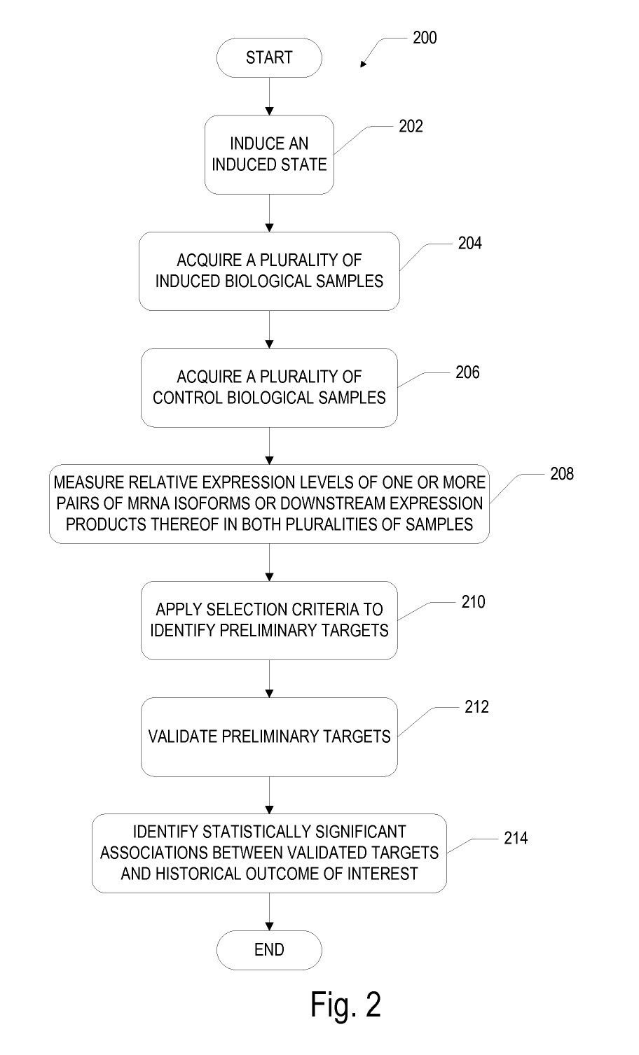

Referring to FIG. 2, this disclosure also provides a method 200 of identifying predictive targets in a cancer of interest. At process block 202, the method 200 can include inducing an induced state in a plurality of inducement subjects. At process block 204, the method 200 can include acquiring a plurality of induced biological samples by acquiring an induced biological sample from each of the plurality of inducement subjects in the induced state. At process block 206, the method 200 can include acquiring a plurality of control biological samples by acquiring a control biological sample from each of the plurality of control subject in a control state. At process block 208, the method 200 can include measuring relative expression levels of one or more pairs of mRNA isoforms or downstream expression products thereof within the plurality of induced biological samples and the plurality of control biological samples and recording the relative expression levels to an expression data set. At process block 210, the method 200 can include applying selection criteria to the expression data set to identify preliminary targets. At process block 212, the method 200 can include validating the preliminary targets resulting in validated targets. At process block 214, the method 200 can include identifying statistically significant associations between the validated targets and an outcome of interest in a historical data set.

In certain aspects, the induced state is a hypoxic state and the inducement subjects are hypoxia subjects. In certain aspects, the induced state is any which reflects a state of cellular stress that can be caused by hypoxia, such as cellular senescence or other physiologic response induced by hypoxia. In certain aspects, the control state is a normoxic state and the control subjects are normoxia subjects.

### Subjects

Suitable subjects can include any living organism capable of having cancer. In certain aspects, the subject can be an animal. In certain aspects, the subject can be a mammal. In certain aspects, the subject can be a primate. In certain aspects, the subject can be a homo sapien.

### Obtaining a Biological Sample

Obtaining a biological sample from a subject can refer to any means of obtaining a sample, including but not limited to, directly obtaining the biological sample from the subject by a variety of means known to those having ordinary skill in the art or indirectly obtaining the biological sample by a variety of means known to those having ordinary skill in the art. Means of directly obtaining the biological sample include, but are not limited to, drawing blood, biopsying a tissue of interest, resecting a tissue of interest, scraping a plurality of cells from a location on the subject, acquiring a stool sample, acquiring a sweat sample, acquiring a saliva sample, acquiring a urine sample, and the like. Means of indirectly obtaining the biological sample include, but are not limited to, receiving a sample from another researcher or lab, receiving a sample from a commercial supplier, retrieving a sample from a storage place, receiving data from processed samples, and the like.

Suitable examples of biological samples can include, but are not limited to, tissues, cells, biopsy samples, blood, lymph, serum, plasma, urine, saliva, mucus, sweat, tears, combinations thereof, and the like. In certain aspects, the biological sample can be a blood sample, such as a plasma sample. In certain aspects, the biological sample can be a biopsy sample, such as a tissue sample or a cell sample.

In certain aspects, more than one biological sample may be obtained from a particular subject, including but not limited to, two or more biological samples, three or more biological samples, four or more biological samples, or five or more biological samples. The acquisition of multiple biological samples from an individual subject can allow the averaging of certain measurements or the comparison between certain induced/control states, as would be understood by a person having ordinary skill in the art.

Control samples can be acquired from control subject. A person having ordinary skill in the art would understand how to identify a control subject and how to acquire a control sample, based on the condition or induced state that is being measured against the control.

### Sample Preparation

After obtaining a biological sample, and prior to any subsequent analysis, certain sample preparation steps may be performed on the biological sample. Typically, these sample preparation operations will include such manipulations as concentration, suspension, extraction of intracellular material, e.g., mRNA from tissue/whole cell samples and the like.

Any method required for the processing of a sample prior to detection by any of the methods noted herein falls within the scope of the present disclosure. These methods are typically well known by a person skilled in the art.

### Measuring a Relative Expression Level

It is within the general scope of the present disclosure to provide methods for measuring a relative expression level of one or more pairs of mRNA isoforms or downstream expression products thereof. An aspect of the present disclosure relates to the detection of one or more pairs of mRNA isoforms or downstream expression products thereof, as described in the specification and figures contained herein.

A person having ordinary skill in the art will appreciate that there are a multitude of ways to measure a relative expression level of pairs of mRNA isoforms or downstream expression products thereof, and the description of particular ways of achieving this measurement is not intended as being limiting to the scope of the invention.

One way to detect a relative expression level of one or more pairs of mRNA isoforms or downstream expression products thereof is to employ the SplicerAV or SplicerEX algorithm as described in Robinson T J, Dinan M A, Dewhirst M, Garcia-Blanco M A, Pearson J L. “SplicerAV: A tool for mining microarray expression data for changes in RNA processing.” BMC Bioinformatics 2010; 11:108 and Robinson T J, Forte E, Salinas R E, et al. “SplicerEX: A tool for the automated detection and classification of mRNA changes from conventional and splice-sensitive microarray expression data.” RNA 2012; 18:1435-45.

The methods described herein can be in situ or screening methods. The term in situ refers to methods of detecting a protein, phosphopeptide, and/or nucleic acid molecule in a sample where the structure of the sample is preserved. In situ methods can include, but are not limited to, in situ hybridization techniques and in situ PCR methods. The term screening method refers to methods requiring preparation of the sample material in order to assess the species to be detected. Screening methods include, but are not limited to, array detection methods, affinity matrices, Northern blotting methods, and PCR techniques such as real-time quantitative RT-PCR.

mRNA Isoforms

This disclosure relates to pairs of mRNA isoforms. Specifically, this disclosure relates to pairs of nRNA isoforms that have varying degrees of expression based on a state of interest, such as, for example, hypoxia.

mRNA isoforms suitable for use with the present invention include, but are not limited to, mRNA isoforms associated with a gene selected from the group consisting of ETNK1, CARD8, TMEM68, ATF7IP, SUV420H1, PPM1B, and ZNF33A, among others.

A pair of mRNA isoforms can include an upregulated isoform and a downregulated isoform, wherein the upregulation and downregulation is relative to a state of interest, such as an induced state, such as hypoxia.

The hypoxia upregulated ETNK1 is the mRNA expressed by SEQ ID NO: 1. The hypoxia downregulated ETNK1 is the mRNA expressed by SEQ ID NO: 2. ETNK1 can be identified by the Affymetrix® transcript cluster ID 3408018.

The hypoxia upregulated CARD8 is the mRNA expressed by SEQ ID NO: 3. The hypoxia downregulated CARD8 is the mRNA expressed by SEQ ID NO: 4. CARD8 can be identified by the Affymetrix® transcript cluster ID 3866958.

The hypoxia upregulated TMEM68 is the mRNA expressed by SEQ ID NO: 5. The hypoxia downregulated TMEM68 is the mRNA expressed by SEQ ID NO: 6. TMEM68 can be identified by the Affymetrix® transcript cluster ID 3136015.

The hypoxia upregulated ATF7IP is the mRNA expressed by SEQ ID NO: 7. The hypoxia downregulated ATF7IP is the mRNA expressed by SEQ ID NO: 8. ATF7IP can be identified by the Affymetrix® transcript cluster ID 3406015.

The hypoxia upregulated SUV420H1 is the mRNA expressed by SEQ ID NO: 9. The hypoxia downregulated SUV420H1 is the mRNA expressed by SEQ ID NO: 10. SUV420H1 can be identified by the Affymetrix® transcript cluster ID 3379390.

The hypoxia upregulated PPM1B is the mRNA expressed by SEQ ID NO: 11. The hypoxia downregulated PPM1B is the mRNA expressed by SEQ ID NO: 12. PPM1B can be identified by the Affymetrix® transcript cluster ID 2479640.

The hypoxia upregulated ZNF33A is the mRNA expressed by SEQ ID NO: 13. The hypoxia downregulated ZNF33A is the mRNA expressed by SEQ ID NO: 14. ZNF33A can be identified by the Affymetrix® transcript cluster ID 3243078.

In certain aspects, the isoform ratio of these pairs of mRNA isoforms can be at least 2, at least 2.1, at least 2.2, at least 2.3, at least 2.4, at least 2.5, at least 2.6, at least 2.7, at least 2.8, at least 2.9, or at least 3.0.

### Primers

One aspect of the present disclosure is to provide a primer which can be used for the PCR or RT-qPCR of one or more of the mRNA isoforms described herein. In certain aspects, the primers described herein can be synthetic primers.

Primers suitable for use with the present invention include, but are not limited to, forward and reverse primers for the mRNA isoforms described herein, such as for the hypoxia upregulated and downregulated mRNA isoforms described herein.

A forward primer for the hypoxia upregulated ETNK1 is represented by SEQ ID NO: 15. A reverse primer for the hypoxia upregulated ETNK1 is represented by SEQ ID NO 16. A forward primer for the hypoxia downregulated ETNK1 is represented by SEQ ID NO: 17. A reverse primer for the hypoxia downregulated ETNK 1 is represented by SEQ ID NO: 18.

A forward primer for the hypoxia upregulated CARD8 is represented by SEQ ID NO: 19. A reverse primer for the hypoxia upregulated CARD8 is represented by SEQ ID NO 20. A forward primer for the hypoxia downregulated CARD8 is represented by SEQ ID NO: 21. A reverse primer for the hypoxia downregulated CARD8 is represented by SEQ ID NO: 22.

A forward primer for the hypoxia upregulated TMEM68 is represented by SEQ ID NO: 23. A reverse primer for the hypoxia upregulated TMEM68 is represented by SEQ ID NO 24. A forward primer for the hypoxia downregulated TMEM68 is represented by SEQ ID NO: 25. A reverse primer for the hypoxia downregulated TMEM68 is represented by SEQ ID NO: 26.

A forward primer for the hypoxia upregulated ATF7IP is represented by SEQ ID NO: 27. A reverse primer for the hypoxia upregulated ATF7IP is represented by SEQ ID NO 28. A forward primer for the hypoxia downregulated ATF7IP is represented by SEQ ID NO: 29. A reverse primer for the hypoxia downregulated ATF7IP is represented by SEQ ID NO: 30.

A forward primer for the hypoxia upregulated SUV420H1 is represented by SEQ ID NO: 31. A reverse primer for the hypoxia upregulated SUV420H1 is represented by SEQ ID NO 32. A forward primer for the hypoxia downregulated SUV420H1 is represented by SEQ ID NO: 33. A reverse primer for the hypoxia downregulated SUV420H1 is represented by SEQ ID NO: 34.

A forward primer for the hypoxia upregulated PPM1B is represented by SEQ ID NO: 35. A reverse primer for the hypoxia upregulated PPM1B is represented by SEQ ID NO 36. A forward primer for the hypoxia downregulated PPM1B is represented by SEQ ID NO: 37. A reverse primer for the hypoxia downregulated PPM1B is represented by SEQ ID NO: 38.

A forward primer for the hypoxia upregulated ZNF33A is represented by SEQ ID NO: 39. A reverse primer for the hypoxia upregulated ZNF33A is represented by SEQ ID NO 40. A forward primer for the hypoxia downregulated ZNF33A is represented by SEQ ID NO: 41. A reverse primer for the hypoxia downregulated ZNF33A is represented by SEQ ID NO: 42.

### Probe

One aspect of the present disclosure is to provide a probe which can be used for the detection of a protein, phosphopeptide, nucleic acid and/or popypeptide molecule as defined herein.

A probe as defined herein is a specific sequence of a nucleic acid and/or polypeptide used to detect nucleic acids and/or polypeptides by hybridization. For example, a nucleic acid is also here any nucleic acid, natural or synthetic such as DNA, RNA, LNA or PNA. A probe may be labeled, tagged or immobilized or otherwise modified according to the requirements of the detection method chosen. A label or a tag is an entity making it possible to identify a compound to which it is associated. It is within the scope of the present disclosure to employ probes that are labeled or tagged by any means known in the art such as but not limited to: radioactive labeling, fluorescent labeling and enzymatic labeling. Furthermore the probe, labeled or not, may be immobilized to facilitate detection according to the detection method of choice and this may be accomplished according to the preferred method of the particular detection method.

A RT-qPCR probe for the hypoxia upregulated ETNK1 is represented by SEQ ID NO: 43. A RT-qPCR probe for the hypoxia downregulated ETNK1 is represented by SEQ ID NO: 44.

A RT-qPCR probe for the hypoxia upregulated CARD8 is represented by SEQ ID NO: 45. A RT-qPCR probe for the hypoxia downregulated CARD8 is represented by SEQ ID NO: 46.

A RT-qPCR probe for the hypoxia upregulated TMEM68 is represented by SEQ ID NO: 47. A RT-qPCR probe for the hypoxia downregulated TMEM68 is represented by SEQ ID NO: 48.

A RT-qPCR probe for the hypoxia upregulated ATF7IP is represented by SEQ ID NO: 49. A RT-qPCR probe for the hypoxia downregulated ATF7IP is represented by SEQ ID NO: 50.

A RT-qPCR probe for the hypoxia upregulated SUV420H1 is represented by SEQ ID NO: 51. A RT-qPCR probe for the hypoxia downregulated SUV420H1 is represented by SEQ ID NO: 52.

A RT-qPCR probe for the hypoxia upregulated PPM1B is represented by SEQ ID NO: 53. A RT-qPCR probe for the hypoxia downregulated PPM1B is represented by SEQ ID NO: 54.

A RT-qPCR probe for the hypoxia upregulated ZNF33A is represented by SEQ ID NO: 55. A RT-qPCR probe for the hypoxia downregulated ZNF33A is represented by SEQ ID NO: 56.

### Detection Methods

Another aspect of the present disclosure regards the detection of nucleic acid and/or polypeptide molecules by methods known to those having ordinary skill in the art. The follow sections describe various detection methods that can be employed for this purpose, and the present disclosure includes all the mentioned methods, but is not limited to any of these.

### In Situ Hybridization

In situ hybridization (ISH) applies and extrapolates the technology of nucleic acid and/or polypeptide hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules. Sample cells are treated to increase their permeability to allow the probe to enter the cells, the probe is added to the treated cells, allowed to hybridize at pertinent temperature, and then excess probe is washed away. A complementary probe is labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

An aspect of the present disclosure includes the method of detection by in situ hybridization as described herein.

### In Situ PCR

In situ PCR is the PCR based amplification of the target nucleic acid sequences prior to ISH. For detection of RNA, an intracellular reverse transcription (RT) step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR the cells are cytocentrifugated onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens. Detection of intracellular PCR-products is achieved by one of two entirely different techniques. In indirect in situ PCR by ISH with PCR-product specific probes, or in direct in situ PCR without ISH through direct detection of labeled nucleotides (e.g. digoxigenin-11-dUTP, fluorescein-dUTP, ³H-CTP or biotin-16-dUTP) which have been incorporated into the PCR products during thermal cycling.

An embodiment of the present disclosure concerns the method of in situ PCR as mentioned herein above for the detection of nucleic acid molecules as detailed herein.

### Microarray

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used e.g. to measure the expression levels of large numbers of polypeptides/proteins/nucleic acids simultaneously.

Microarrays can be fabricated using a variety of technologies, including printing with fine-pointed pins onto glass slides, photolithography using pre-made masks, photolithography using dynamic micromirror devices, ink jet printing, or electrochemistry on microelectrode arrays.

An aspect of the present disclosure regards the use of microarrays for the expression profiling of biomarkers for determining whether a tumor is hypoxic and/or whether a cancer/tumor will be susceptible to a hypoxia-specific treatment. For this purpose, and by way of example, mRNA is extracted from a cell or tissue sample, the small RNAs (18-26-nucleotide RNAs) are size-selected from total RNA using denaturing polyacrylamide gel electrophoresis (PAGE). Then oligonucleotide linkers are attached to the 5′ and 3′ ends of the small RNAs and the resulting ligation products are used as templates for an RT-PCR reaction with 10 cycles of amplification. The sense strand PCR primer has a Cy3 fluorophore attached to its 5′ end, thereby fluorescently labelling the sense strand of the PCR product. The PCR product is denatured and then hybridized to the microarray. A PCR product, referred to as the target nucleic acid that is complementary to the corresponding RNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular biomarker, using a number of positive and negative controls and array data normalization methods, which will result in assessment of the level of expression of a particular biomarker.

Several types of microarrays can be employed such as spotted oligonucleotide microarrays, pre-fabricated oligonucleotide microarrays or spotted long oligonucleotide arrays. In spotted oligonucleotide microarrays the capture probes are oligonucleotides complementary to nucleic acid sequences. This type of array is typically hybridized with amplified. PCR products of size-selected small RNAs from two samples to be compared that are labelled with two different fluorophores. Alternatively, total RNA containing the small RNA fraction is extracted from the abovementioned two samples and used directly without size-selection of small RNAs, and 3′ end labeled using T4 RNA ligase and short RNA linkers labelled with two different fluorophores. The samples can be mixed and hybridized to one single microarray that is then scanned, allowing the visualization of up-regulated and down-regulated biomarker genes in one go. The downside of this is that the absolute levels of gene expression cannot be observed, but the cost of the experiment is reduced by half. Alternatively, a universal reference can be used, comprising of a large set of fluorophore-labelled oligonucleotides, complementary to the array capture probes.

In pre-fabricated oligonucleotide microarrays or single-channel microarrays, the probes are designed to match the sequences of known or predicted biomarkers. There are commercially available designs that cover complete genomes from companies such as Affymetrix, or Agilent. These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing. More recently, Maskless Array Synthesis from NimbleGen Systems has combined flexibility with large numbers of probes. Arrays can contain up to 390,000 spots, from a custom array design.

An embodiment of the present disclosure concerns the method of microarray use and analysis as described herein.

### PCR

The terms “PCR reaction”, “PCR amplification”, “PCR”, “pre-PCR”, “Q-PCR”, “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence based amplification and Q Beta Replicase systems. The products formed by said amplification reaction may or may not be monitored in real time or only after the reaction as an end-point measurement.

### Real-Time Quantitative RT-PCR

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This is commonly used for the purpose of determining whether a genetic sequence is present or not, and if it is present the number of copies in the sample. There are 3 methods which vary in difficulty and detail. Like other forms of polymerase chain reaction, the process is used to amplify DNA samples, using thermal cycling and a thermostable DNA polymerase.

The three commonly used methods of quantitative polymerase chain reaction are through agarose gel electrophoresis, the use of SYBR Green, a double stranded DNA dye, and the fluorescent reporter probe. The latter two of these three can be analysed in real-time, constituting real-time polymerase chain reaction method.

Agarose gel electrophoresis is the simplest method, but also often slow and less accurate then other methods, depending on the running of an agarose gel via electrophoresis. It cannot give results in real time. The unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown. This method is generally used as a simple measure of whether the probe target sequences are present or not, and rarely as ‘true’ Q-PCR.

Using SYBR Green dye is more accurate than the gel method, and gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artefacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescent reporter probe method is the most accurate and most reliable of the methods. It uses a sequence-specific nucleic acid based probe so as to only quantify the probe sequence and not all double stranded DNA. It is commonly carried out with DNA based probes with a fluorescent reporter and a quencher held in adjacent positions, so-called dual-labelled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerisation continues it reaches the probe bound to its complementary sequence, which is then hydrolysed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolysed dual-labelled probe in each PCR cycle is monitored, which allows accurate determination of the final, and so initial, quantities of DNA.

Any method of PCR that can determine the expression of a nucleic acid molecule as defined herein falls within the scope of the present disclosure. A preferred aspect of the present disclosure includes the real-time quantitative RT-PCR method, based on the use of either SYBR Green dye or a dual-labeled probe for the detection and quantification of nucleic acids according to the herein described.

### Northern Blot Analysis

An aspect of the present disclosure includes the detection of the nucleic acid molecules herein disclosed by techniques such as Northern blot analysis. Many variations of the protocol exist.

### Immunohistochemistry

Immunohistochemistry, or IHC, refers to the process of detecting antigens (e.g., proteins) in cells of a tissue section by exploiting the principle of antibodies binding specifically to antigens in biological tissues. Such staining is widely used in the diagnosis of abnormal cells such as those found in cancerous tumors. One aspect of the present disclosure relates to the identification of hypoxic-associated changes in the biomarkers provided herein (e.g., mRNA processing that results in protein coding changes). Therefore, according to some embodiments, the detection comprises immunohistochemistry. In certain embodiments, the identification of hypoxic-associated changes in the biomarkers provided herein (i.e., hypoxic-associated changes in mRNA processing that results in protein coding changes) are identified using immunohistochemistry with (1) isoform specific antibodies; (2) by changes in subcellular and intracellular localization; or (3) by changes in protein product size by Western blot. The results of such detection may then be used to identify regions of hypoxia and hence therapeutic, prognostic, and diagnostic information obtained from the tumor samples.

### Selection Criteria to Identify Preliminary Targets

In certain aspects, the methods described herein can include applying selection criteria to identify preliminary targets. The selection criteria can include one or more of the following: limiting hits to those with alternative splicing fold changes that are greater than 2; limiting the hits to isoform ratio fold changes of greater than 2; limiting the hits to an isoform ratio p-value of less than or equal to 0.0001; limiting the hits to alternative splicing changes that are predicted to result in protein coding changes; and combinations thereof.

### Validating Preliminary Targets

In certain aspects, the methods can include validating the preliminary targets described herein. Methods of validating the preliminary targets can include, but are not limited to, RT-qPCR, Immunohistochemistry, Western blot, Northern blot, 3′ RACE, generally any method that can be used to differentiate the relative abundance of two mRNA isoforms or resulting protein products, or a combination thereof.

### Historical or Contemporary Data Set

In certain aspects, the historical data set described herein can be any data set relating to a cancer of interest, which has sufficient data for comparison of relative expression levels of mRNA isoforms as described herein. In certain aspects, the historical data set can include a survival rate, including but not limited to, a survival rate without relapse.

Examples of historical data sets are the data sets for the Lee and Okayama cohorts described below in Example 2.

In certain aspects, the contemporary data set described herein can be any data set relating to a cancer of interest, which has sufficient data for comparison of relative expression levels of mRNA isoforms as described herein.

Examples of contemporary data sets are data sets that are acquired at roughly the same time as a biological sample is acquired from the subject that is the target of the method.

It should be appreciated that the distinction between historical and contemporary is purely time-based, and that in certain aspects of the present disclosure, a single data set could be considered historical and contemporary in differing contexts.

### Prognosis

In certain aspects, the methods described herein can include assessing, varying, altering, or providing a prognosis of a subject suffering from an adenocarcinoma.

In certain aspects, the prognosis is a good prognosis if the relative expression level of a hypoxia upregulated isoform of ATF7IP and a hypoxia downregulated isoform of ATF7IP is increased compared to the relative historical or contemporary cohort expression levels of the hypoxia upregulated isoform of ATF7IP and the hypoxia downregulated isoform of ATF7IP of the historical or contemporary data set. In certain aspects, the prognosis is a poor prognosis if the relative expression level of a hypoxia upregulated isoform of ATF7IP and a hypoxia downregulated isoform of ATF7IP is decreased compared to the relative historical or contemporary cohort expression levels of the hypoxia upregulated isoform of ATF7IP and the hypoxia downregulated isoform of ATF7IP of the historical or contemporary data set. In certain aspects, the prognosis is a good prognosis if the relative expression level of a hypoxia upregulated isoform of ATF7IP and a hypoxia downregulated isoform of ATF7IP is greater than 50%, greater than 55%, greater than 60%, greater than 65%, greater than 70%, greater than 75%, greater than 80%, greater than 85%, or greater than 90% of the relative historical or contemporary cohort expression levels of the hypoxia upregulated isoform of ATF7IP and the hypoxia downregulated isoform of ATF7IP of the historical or contemporary data set. In certain aspects, the prognosis is a poor prognosis if the relative expression level of a hypoxia upregulated isoform of ATF7IP and a hypoxia downregulated isoform of ATF7IP is less than 50%, less than 55%, less than 60%, less than 65%, less than 70%, less than 75%, less than 80%, less than 85%, or less than 90% of the relative historical or contemporary cohort expression levels of the hypoxia upregulated isoform of ATF7IP and the hypoxia downregulated isoform of ATF7IP of the historical or contemporary data set.

In certain aspects, the prognosis is a good prognosis if the relative expression level of a hypoxia upregulated isoform of PPM1B and a hypoxia downregulated isoform of PPM1B is decreased compared to the relative historical or contemporary cohort expression levels of the hypoxia upregulated isoform of PPM1B and the hypoxia downregulated isoform of PPM1B of the historical or contemporary data set. In certain aspects, the prognosis is a poor prognosis if the relative expression level of a hypoxia upregulated isoform of PPM and a hypoxia downregulated isoform of PPM is increased compared to the relative historical or contemporary cohort expression levels of the hypoxia upregulated isoform of PPM and the hypoxia downregulated isoform of PPM of the historical or contemporary data set. In certain aspects, the prognosis is a good prognosis if the relative expression level of a hypoxia upregulated isoform of PPM1B and a hypoxia downregulated isoform of PPM1B is less than 50%, less than 55%, less than 60%, less than 65%, less than 70%, less than 75%, less than 80%, less than 85%, or less than 90% of the relative historical or contemporary cohort expression levels of the hypoxia upregulated isoform of PPM and the hypoxia downregulated isoform of PPM1B of the historical or contemporary data set. In certain aspects, the prognosis is a poor prognosis if the relative expression level of a hypoxia upregulated isoform of PPM1B and a hypoxia downregulated isoform of PPM1B is greater than 50%, greater than 55%, greater than 60%, greater than 65%, greater than 70%, greater than 75%, greater than 80%, greater than 85%, or greater than 90% of the relative historical or contemporary cohort expression levels of the hypoxia upregulated isoform of PPM1B and the hypoxia downregulated isoform of PPM1B of the historical or contemporary data set.

In certain aspects, the prognosis is a good prognosis if the relative expression level of a hypoxia upregulated isoform of ETNK1 and a hypoxia downregulated isoform of ETNK1 is decreased compared to the relative historical or contemporary cohort expression levels of the hypoxia upregulated isoform of ETNK1 and the hypoxia downregulated isoform of ETNK1 of the historical or contemporary data set. In certain aspects, the prognosis is a poor prognosis if the relative expression level of a hypoxia upregulated isoform of ETNK1 and a hypoxia downregulated isoform of ETNK1 is increased compared to the relative historical or contemporary cohort expression levels of the hypoxia upregulated isoform of ETNK1 and the hypoxia downregulated isoform of ETNK1 of the historical or contemporary data set. In certain aspects, the prognosis is a good prognosis if the relative expression level of a hypoxia upregulated isoform of ETNK1 and a hypoxia downregulated isoform of ETNK1 is less than 50%, less than 55%, less than 60%, less than 65%, less than 70%, less than 75%, less than 80%, less than 85%, or less than 90% of the relative historical or contemporary cohort expression levels of the hypoxia upregulated isoform of ETNK1 and the hypoxia downregulated isoform of ETNK1 of the historical or contemporary data set. In certain aspects, the prognosis is a poor prognosis if the relative expression level of a hypoxia upregulated isoform of ETNK1 and a hypoxia downregulated isoform of ETNK1 is greater than 50%, greater than 55%, greater than 60%, greater than 65%, greater than 70%, greater than 75%, greater than 80%, greater than 85%, or greater than 90% of the relative historical or contemporary cohort expression levels of the hypoxia upregulated isoform of ETNK1 and the hypoxia downregulated isoform of ETNK1 of the historical or contemporary data set.

In certain aspects, the prognosis is a good prognosis if the relative expression level of a hypoxia upregulated isoform of CARD8 and a hypoxia downregulated isoform of CARD8 is decreased compared to the relative historical or contemporary cohort expression levels of the hypoxia upregulated isoform of CARD8 and the hypoxia downregulated isoform of CARD8 of the historical or contemporary data set. In certain aspects, the prognosis is a poor prognosis if the relative expression level of a hypoxia upregulated isoform of CARD8 and a hypoxia downregulated isoform of CARD8 is increased compared to the relative historical or contemporary cohort expression levels of the hypoxia upregulated isoform of CARD8 and the hypoxia downregulated isoform of CARD8 of the historical or contemporary data set. In certain aspects, the prognosis is a good prognosis if the relative expression level of a hypoxia upregulated isoform of CARD8 and a hypoxia downregulated isoform of CARD8 is less than 50%, less than 55%, less than 60%, less than 65%, less than 70%, less than 75%, less than 80%, less than 85%, or less than 90% of the relative historical or contemporary cohort expression levels of the hypoxia upregulated isoform of CARD8 and the hypoxia downregulated isoform of CARD8 of the historical or contemporary data set. In certain aspects, the prognosis is a poor prognosis if the relative expression level of a hypoxia upregulated isoform of CARD8 and a hypoxia downregulated isoform of CARD8 is greater than 50%, greater than 55%, greater than 60%, greater than 65%, greater than 70%, greater than 75%, greater than 80%, greater than 85%, or greater than 90% of the relative historical or contemporary cohort expression levels of the hypoxia upregulated isoform of CARD8 and the hypoxia downregulated isoform of CARD8 of the historical or contemporary data set.

### Inducing Switch in Isoforms

In certain aspects, the methods described herein can further include inducing a change in the relative population of a pair of isoforms, such that an isoform associated with a good prognosis is increased in relative population and an isoform associated with a poor prognosis is decreased in relative population. For example, the methods can include one or more of the following: inducing an increase in the isoform ratio of ATF7IP; or inducing a decrease in the isoform ratio of PPM1B, ETNK1, or CARD8.

### Cancers

Methods described herein that are not limited to a specific type of cancer can relate to cancers that are selected from the group consisting of head and neck cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and combinations thereof.

In aspects of the invention relating to adenocarcinoma, the adenocarcinoma can be adenocarcinoma of the esophagus, adenocarcinoma of the pancreas, adenocarcinoma of the prostate, adenocarcinoma of the cervix, adenocarcinoma of the stomach, adenocarcinoma of the breast, adenocarcinoma of the colon, adenocarcinoma of the small bowel or duodenum, adenocarcinoma of the lung, cholangiocarcinoma, adenocarcinoma of the vagina, adenocarcinoma of the ovary, adenocarcinoma of the salivary gland, adenocarcinoma of the uterus, adenocarcinoma of the cervix, or adenocarcinoma of the urachus.

### Treatments

In certain aspects, the treatments described herein can be treatments known to those having ordinary skill in the art to be effective for treating a cancer as described herein.

In certain aspects, the treatments described herein can be hypoxic-specific treatments. Such treatments may comprise administering compounds which are active in inducing hypoxic cell death. For example, prodrugs such as SC30000 induce hypoxic cell death by being broken down into relatively nontoxic reduced metabolites that bind irreversibly to protein thiols inside hypoxic cells. Therefore, in certain embodiments, hypoxic specific treatments may comprise administering a therapeutically effective amount of a compound such as nimorazole, tirapazamine, TH302, SN30000, PR-104, AQ4N, and combinations thereof (see, e.g., Chitneni, SK et al. (2013), Society of Nucelar Medicine 54(8):1339-46). In some aspects, the hypoxia-specific treatments are administered prior to or concurrent with other conventional anti-cancer treatments. Such administration is specific to the subject and can be determined by one skilled in the art at the time of administration.

In certain aspects, the treatments described herein can include administering to a subject suffering from an adenocarcinoma a therapeutically effective amount of a compound selected from the group consisting of nimorazole, tirapazamine, TH302, SN30000, PR-104, AQ4N, and combinations thereof.

Administering treatment to a subject can be a local administration or a systemic administration. The administering can be achieved by, for example, oral administration, subcutaneous injection, intravenous injection, topical administration, implant, targeted local (i.e. intra-tumoral or peri-tumoral) injection of therapy, or other administering methods known to those having ordinary skill in the art.

In certain aspects, the prognosis provided by the methods described herein can be used to indicate that a general more aggressive treatment strategy should be pursued and the methods can include implementing the general more aggressive treatment strategy. In certain aspects, the general more aggressive treatment strategy can include adjuvant, neoadjuvant, or perioperative chemotherapy, neoadjuvant or postoperative radiation therapy, increased surveillance, increased extent of surgery, or any other method of escalating additional cancer-directed care.

### Kits

In certain aspects, this disclosure provides kits. The kits can include the following: an apparatus, a composition of matter, or a combination thereof for measuring a relative expression level of one or more pairs of mRNA isoforms or downstream expression products thereof within a biological sample; and an indication of the impact that the relative expression level has on a prognosis. The indication can be in the form of a visual chart, a computer program, and the like.

### EXAMPLES

### Example 1

### Prospective Hits

SplicerEX was applied to Affymetrix® U133 plus 2 gene expression data obtained from an in vivo hypoxia model of A549 lung adenocarcinoma xenografts in which mice were exposed to ambient (20% O₂, N=5) vs. chronic hypoxic (10% O₂, N=5) breathing conditions for 24 hours followed by immediate sacrifice. SplicerEX detected mRNA isoform changes from this model of hypoxia were subsequently examined for recurrence in a previously published clinical dataset of gene expression from 253 human lung adenocarcinomas.

A total of 214 genes underwent changes in alternative mRNA processing in hypoxic vs. normoxic xenograft tumors (P<0.01). Of the 61 genes predicted to result in truncated protein products, 8 of these genes underwent changes in mRNA isoform, with increased expression of the truncated vs. full length mRNA associated with early recurrence in a clinical lung adenocarcinoma cohort after bonferroni correction for multiple hypothesis testing (TTLL5, LAMC2, EHMT2, ATF71P, EXOC7, NT5E, KLHLS, POLR3E; all P<0.05). Changes in alternative mRNA isoforms associated with each of these genes may be confirmed in clinical samples via RT-PCT, microarray analysis, or immunohistochemistry.

### Example 2

### Additional Prospective Hits and Validating Hits

The same experimental conditions from Example 1 were utilized in this Example. As described above, SplicerEX identified 214 alternative splicing events in the mouse tumors treated with hypoxia versus normoxia. A set of preliminary targets were identified using the following criteria: 1) limiting the hits to those with alternative splicing fold changes that are greater than 2 (“UP” isoform vs. “DOWN” isoform)—N=214; 2) limiting the hits to an isoform ratio (IR) fold change of greater than 2—N=24; 3) limiting the hits to an IR p-value of less than or equal to 0.0001—N=14; and limiting the hits to AS changes predicted to result in protein coding changes—N=8. The set of preliminary targets were identified as ETNK1, CARD8, TMEM68, NUP160, ATF7IP, SUV420H1, PPM1B, and ZNF33A. The isoform data is shown below in Table 1. UpFold=fold increase in the “UP” isoform. UpPval=p-value of UpFold. DownFold=fold increase in the “DOWN” isoform. DownPval=p-value of DownFold. IR_Fold=UpFold/DownFold. IR_Pval=p-value of IR_Fold.

TABLE 1

| GeneSymbol | TID | UpFold | UpPval | DownFold | DownPval | IR_Fold | IR_Pval |
|---|---|---|---|---|---|---|---|
| ETNK1 | 3408018 | 2.388 | 1.73E−05 | 0.761 | 0.006658 | 3.137 | 0 |
| CARD8 | 3866958 | 1.562 | 2.67E−05 | 0.531 | 4.47E−05 | 2.941 | 0 |
| TMEM68 | 3136015 | 1.392 | 0.001333 | 0.602 | 0.000425 | 2.311 | 0.00012 |
| NUP160 | 3371986 | 1.502 | 0.000103 | 0.661 | 0.003934 | 2.272 | 0.00001 |
| ATF7IP | 3406015 | 1.187 | 0.016145 | 0.561 | 7.72E−06 | 2.117 | 0.00008 |
| SUV420H1 | 3379390 | 1.821 | 3.10E−05 | 0.894 | 0.014388 | 2.038 | 0.00001 |
| PPM1B | 2479640 | 1.627 | 0.000447 | 0.801 | 0.000572 | 2.032 | 0.00009 |
| ZNF33A | 3243078 | 1.474 | 0.000501 | 0.731 | 9.72E−05 | 2.018 | 0.0001 |

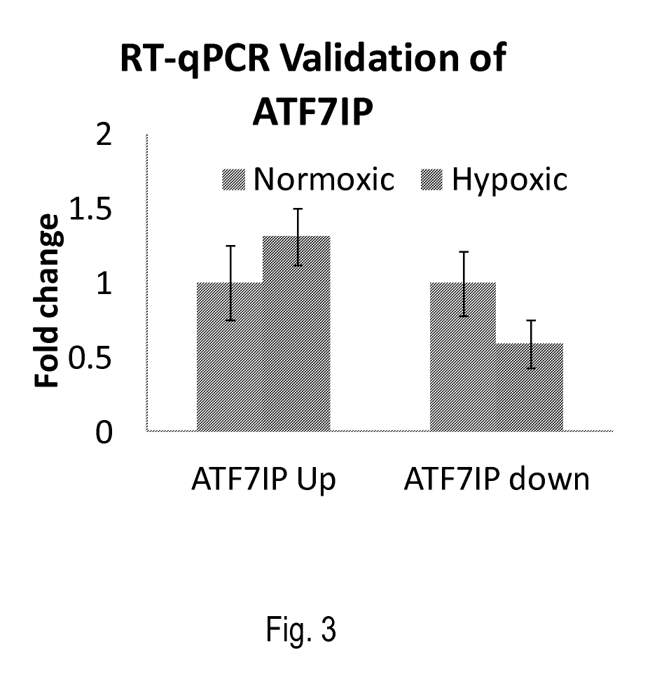

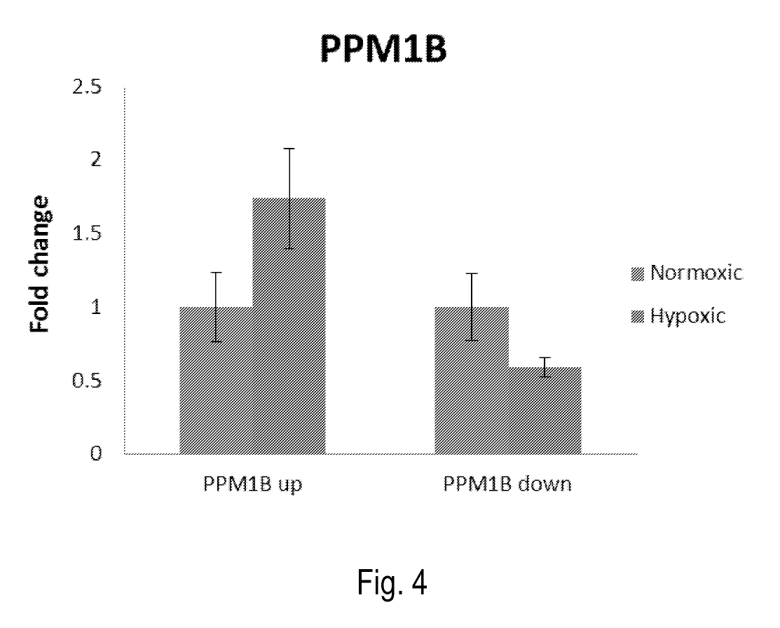

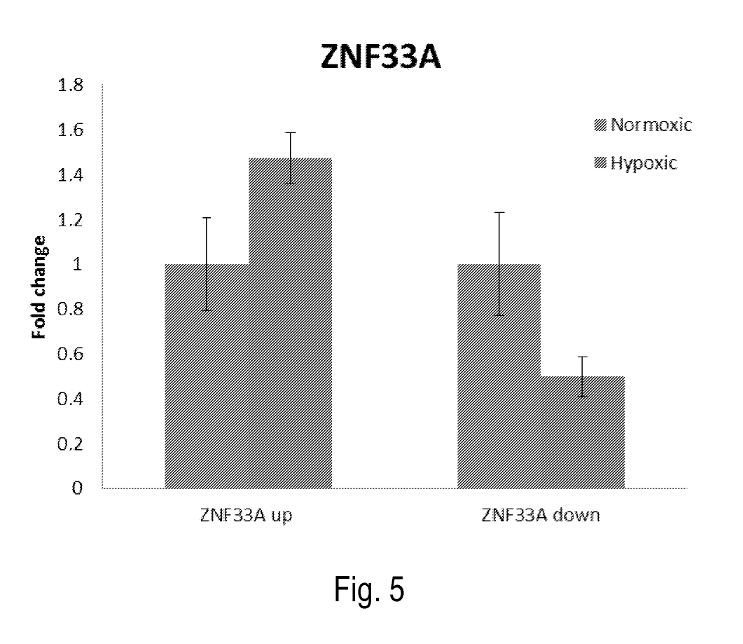

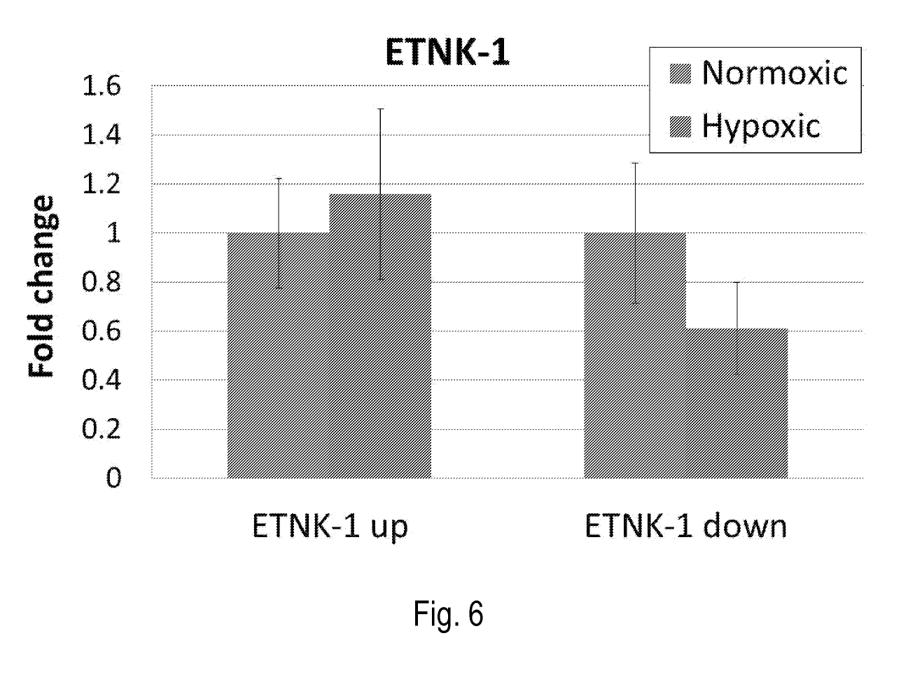

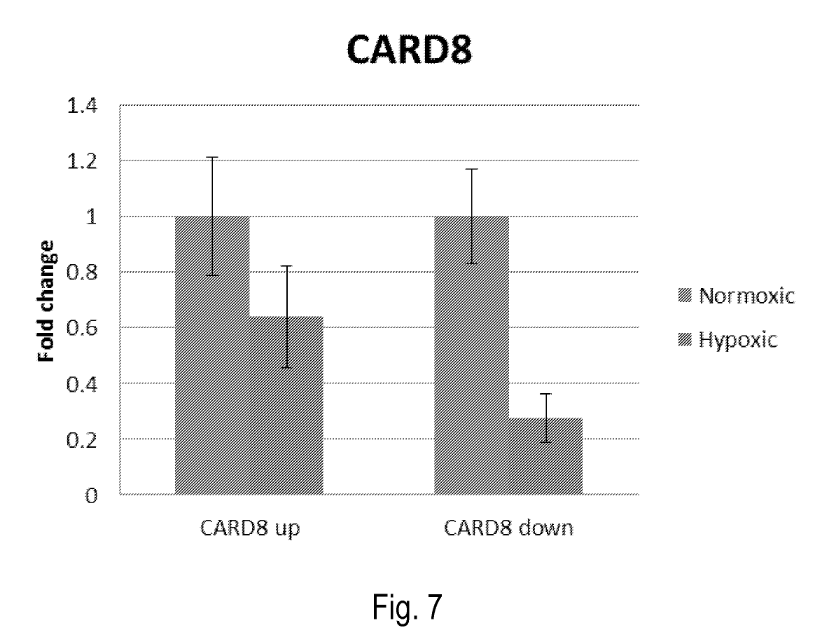

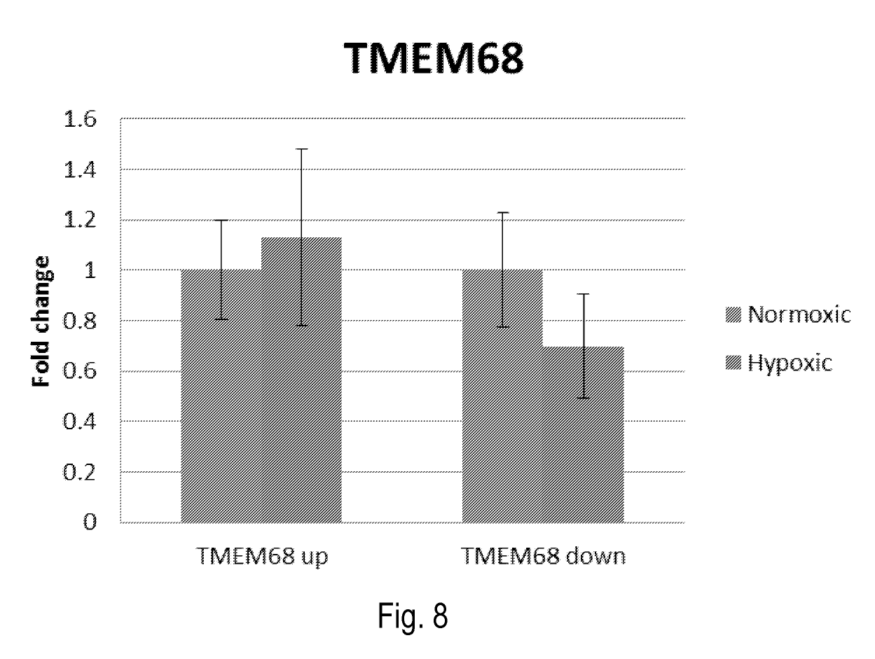

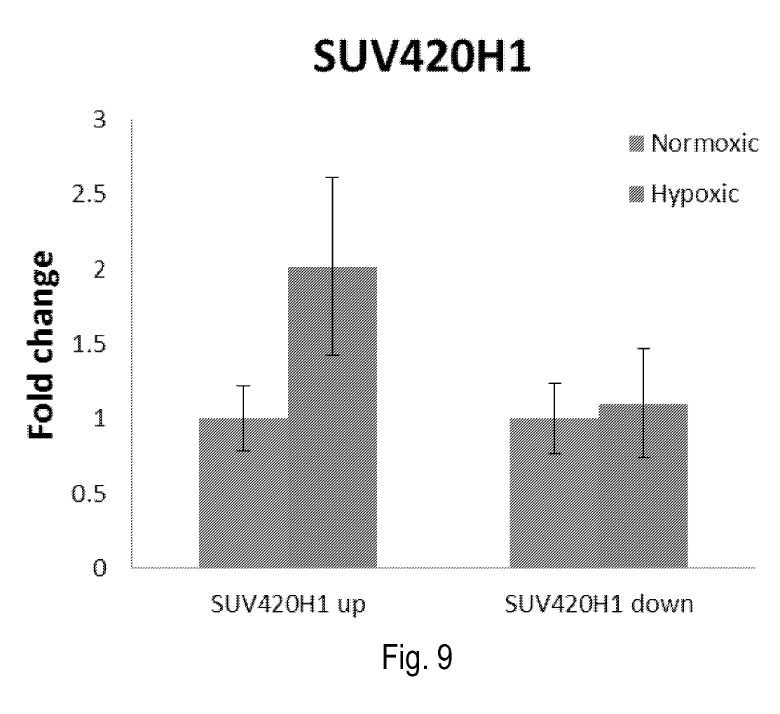

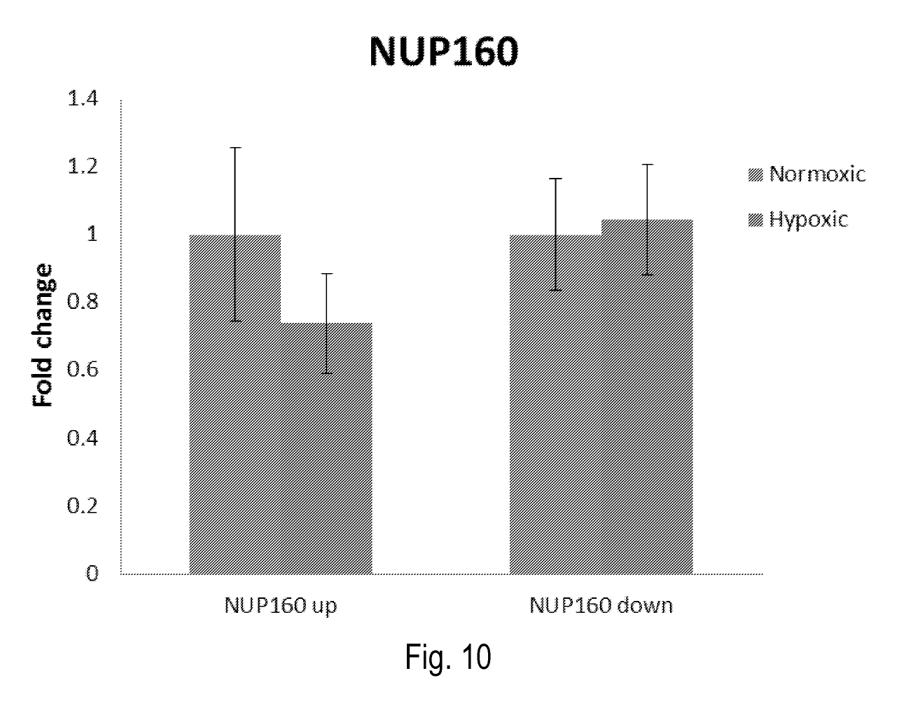

The 8 preliminary targets where then tested using RT-qPCR. 7 of the 8 preliminary targets were validated, with only NUP160 failing to validate. FIG. 3 is a plot showing the RT-qPCR validation data for ATF7IP. FIG. 4 is a plot showing the RT-qPCR validation data for PPM1B. FIG. 5 is a plot showing the RT-qPCR validation data for ANF33A. FIG. 6 is a plot showing the RT-qPCR validation data for ETNK1. FIG. 7 is a plot showing the RT-qPCR validation data for CARD8. FIG. 8 is a plot showing the RT-qPCR validation data for TMEM68. FIG. 9 is a plot showing the RT-qPCR validation data for SUV420H1. FIG. 10 is a plot showing the RT-qPCR validation data for NUP160, which is the only preliminary target that failed to show any reproducible change in isoform abundance with exposure to hypoxia.

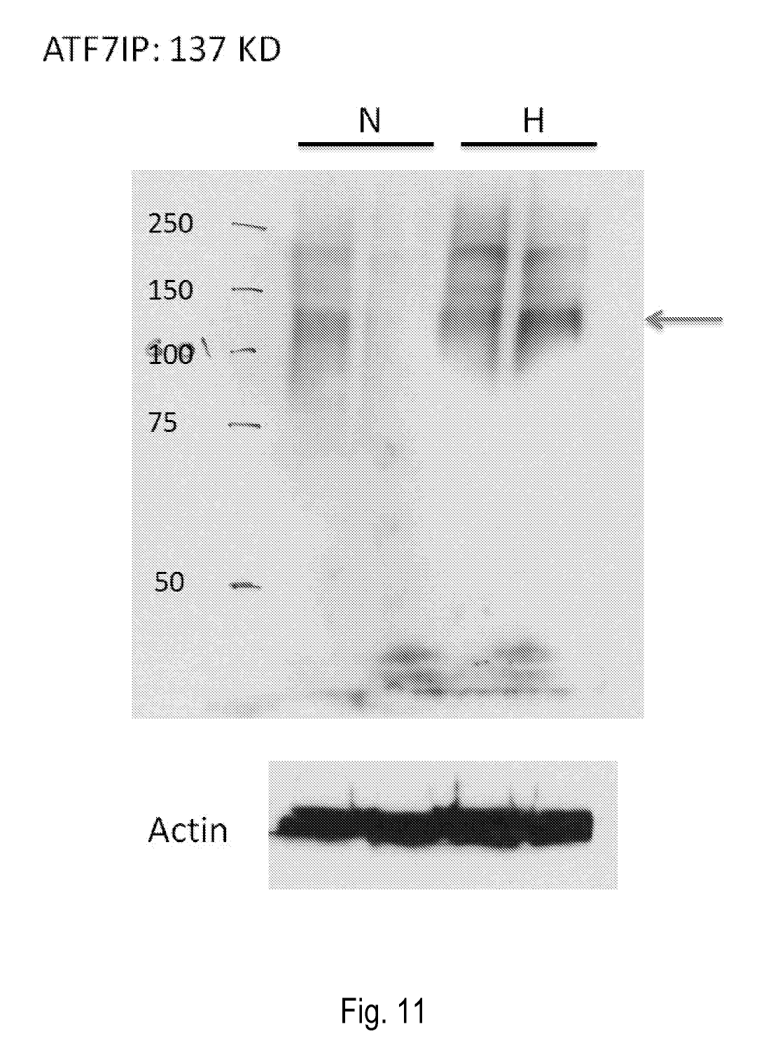

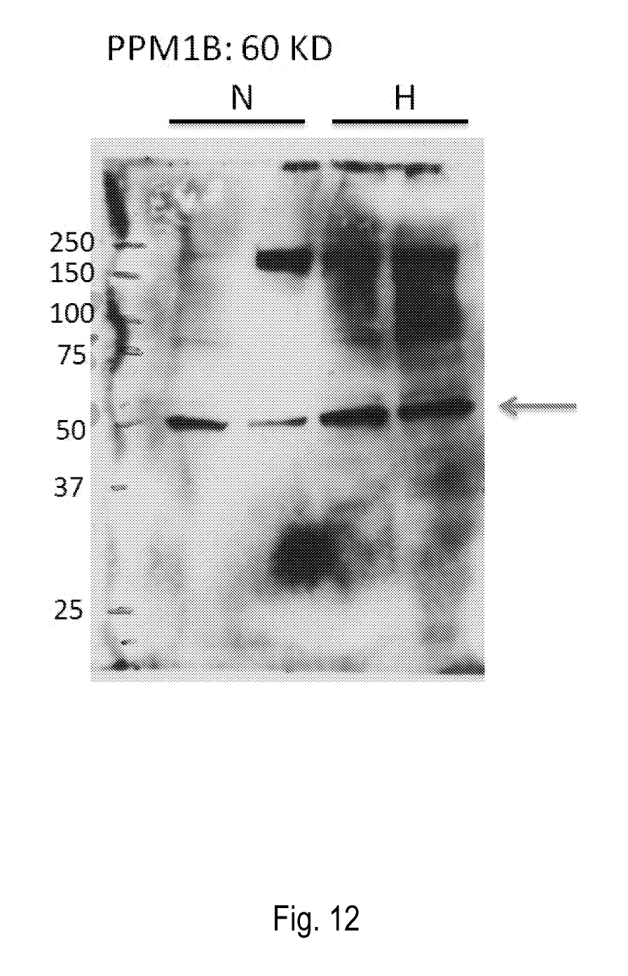

Western blots were performed for ATF7IP and PPM1B, and are shown in FIGS. 11 and 12, respectively. Both Western blots demonstrated an increase of the expressed protein in hypoxia when two normoxic controls were compared with two hypoxic controls.

The 7 validated genes were then investigated for an associate with relapse-free survival in patients with early stage adenocarcinoma of the lung. Two data sets were used for comparison: one from Lee et al., Clin Cancer Res 2008; 14(22), Nov. 15, 2008 (“the Lee cohort”); and another from Okayama et al., Cancer Res; 72(1), Jan. 1, 2012 (“the Okayama cohort”). The Lee cohort included 63 subject having adenocarcinoma of the lung. The Okayama cohort included 58 subjects having adenocarcinoma of the lung. It should be appreciated that data for the Lee cohort and the Okayama cohort both include Affymetrix® U133 plus 2 gene expression data. Of the 7 validated genes, 4 showed significant association with survival in both cohorts.

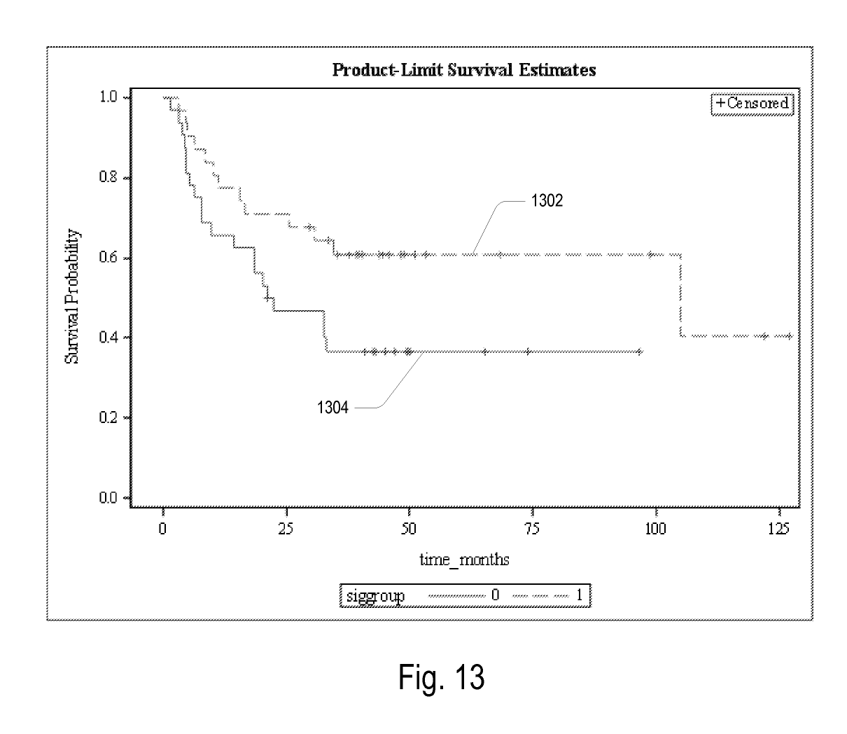

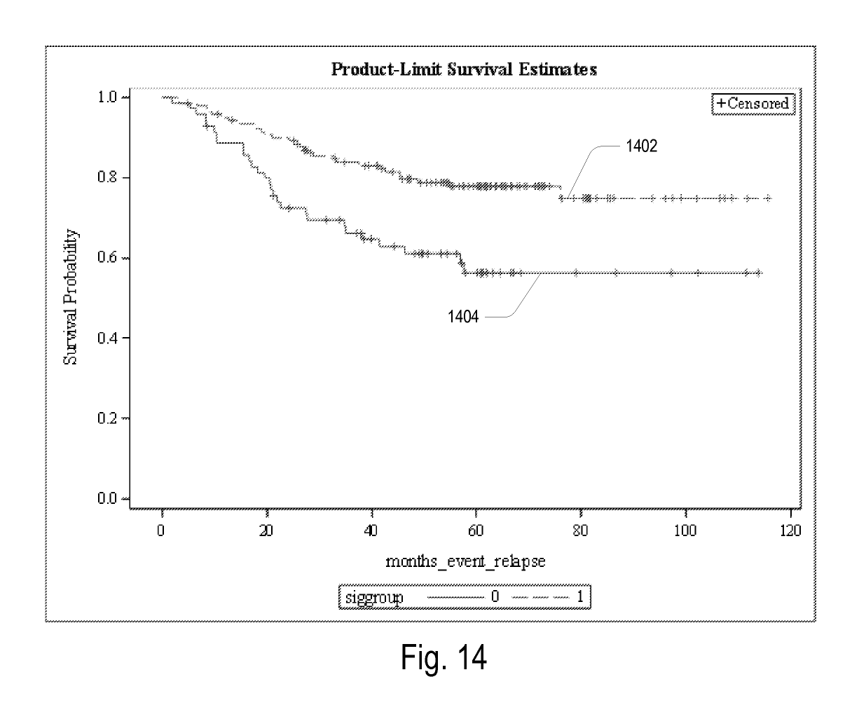

The hypoxic upregulated isoform of ATF7IP was associated with good prognosis in both cohorts. Patients having a top 50th percentile isoform ratio had superior survival rates compared with patients having the lower 50th percentile isoform ratio. FIG. 13 is a plot of the survival probability versus time for patients from the Lee cohort having a top 50th percentile isoform ratio (1302) and patients having a bottom 50th percentile isoform ratio (1304). FIG. 14 is a plot of the survival probability versus time for patients from the Okayama cohort having a top 50th percentile isoform ratio (1402) and patients having a bottom 50th percentile isoform ratio (1404).

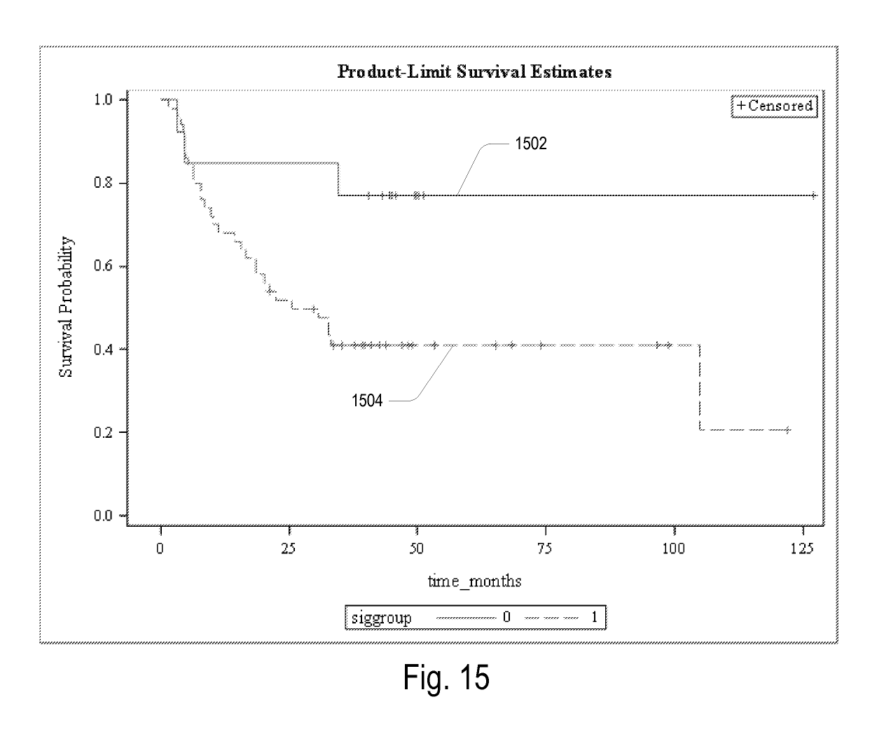

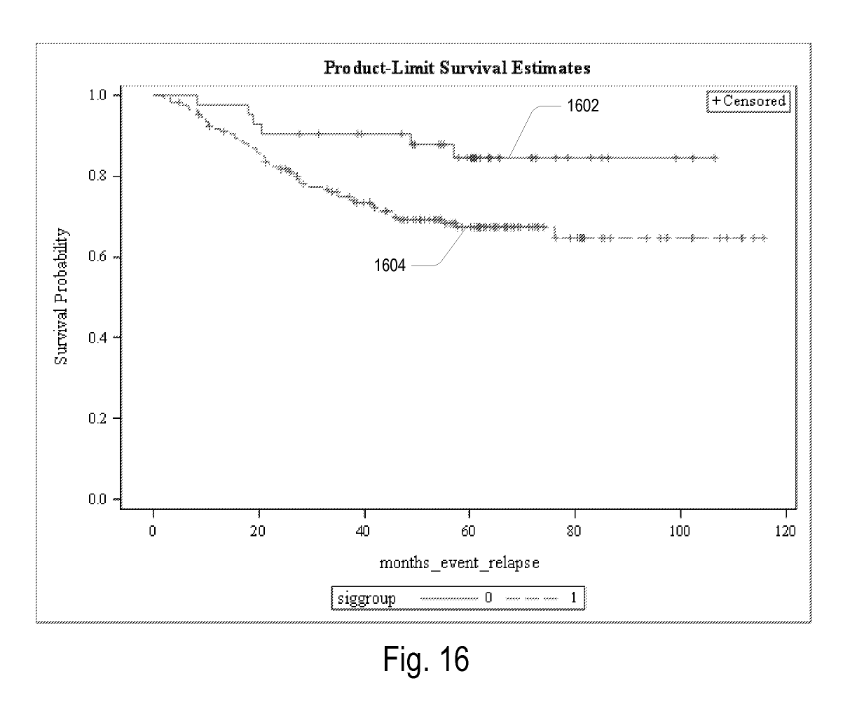

The hypoxic downregulated isoform of PPM1B was associated with poor prognosis in both cohorts. Patients having a top 80th percentile isoform ratio had a worse prognosis compared with patients having the bottom 20th percentile isoform ratio. FIG. 15 is a plot of the survival probability versus time for patients from the Lee cohort having a bottom 20th percentile isoform ratio (1502) and patients having a top 80th percentile isoform ratio (1504). FIG. 16 is a plot of the survival probability versus time for patients from the Okayama cohort having a bottom 20th percentile isoform ratio (1602) and patients having a top 80th percentile isoform ratio (1604).

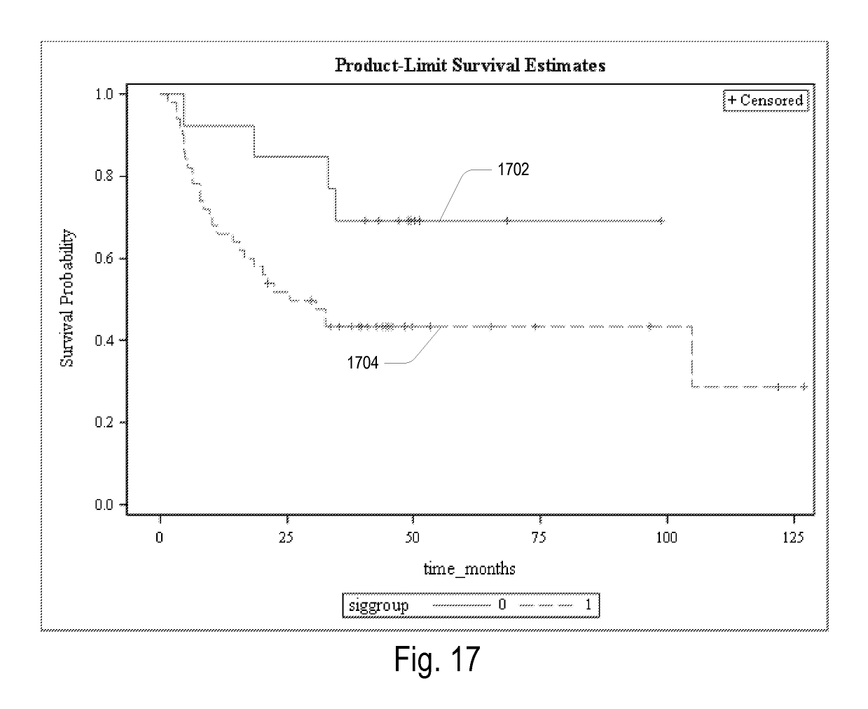

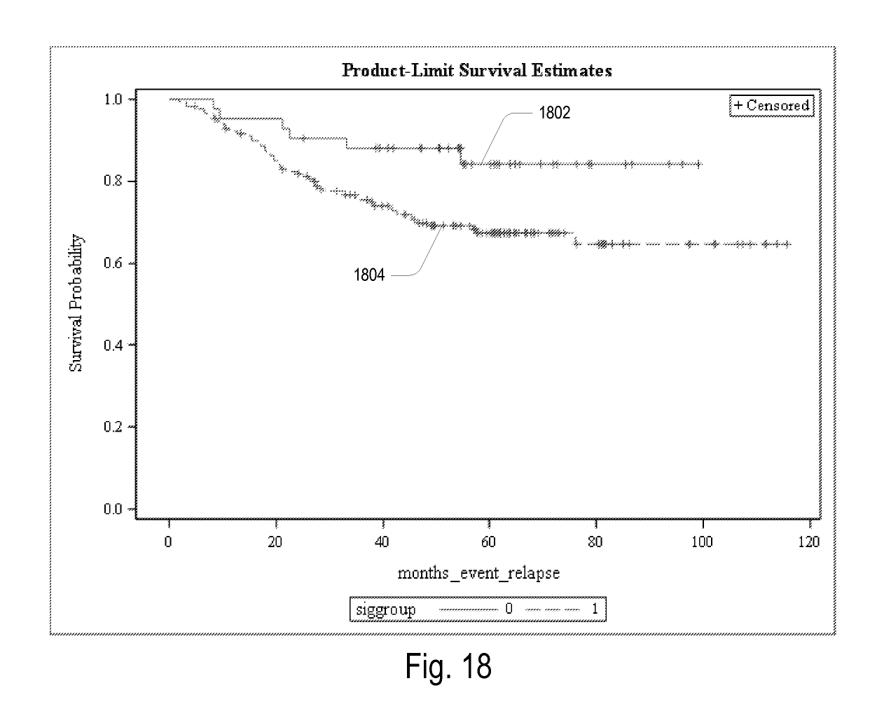

The hypoxic downregulated isoform of ETNK1 was associated with poor prognosis in both cohorts. Patients having a top 80th percentile isoform ratio had a worse prognosis compared with patients having the bottom 20th percentile isoform ratio for each of PPM1B and ETNK1. FIG. 17 is a plot of the survival probability versus time for patients from the Lee cohort having a bottom 20th percentile isoform ratio (1702) and patients having a top 80th percentile isoform ratio (1704). FIG. 18 is a plot of the survival probability versus time for patients from the Okayama cohort having a bottom 20th percentile isoform ratio (1802) and patients having a top 80th percentile isoform ratio (1804).

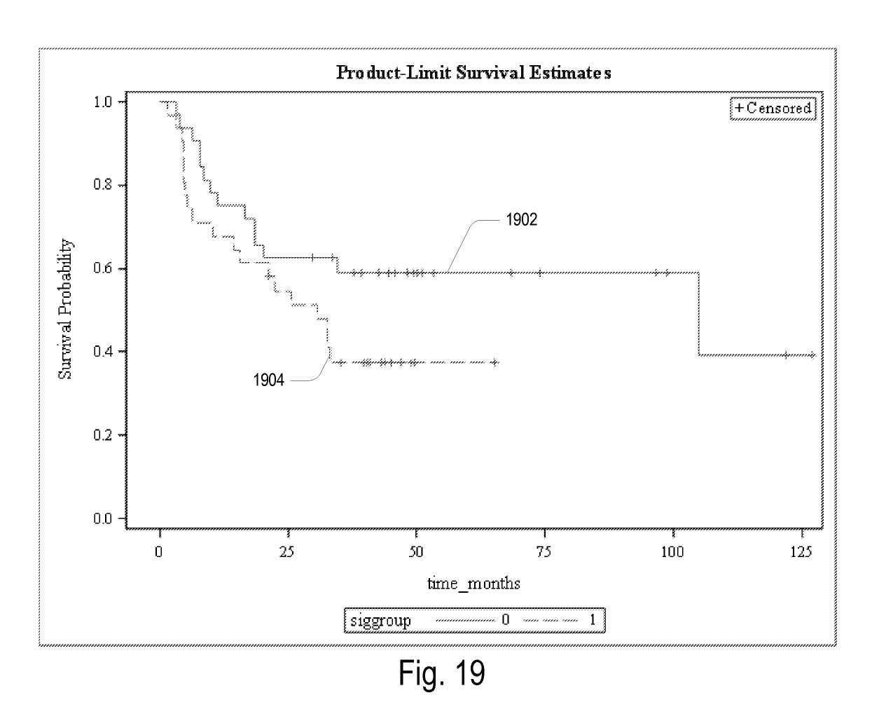

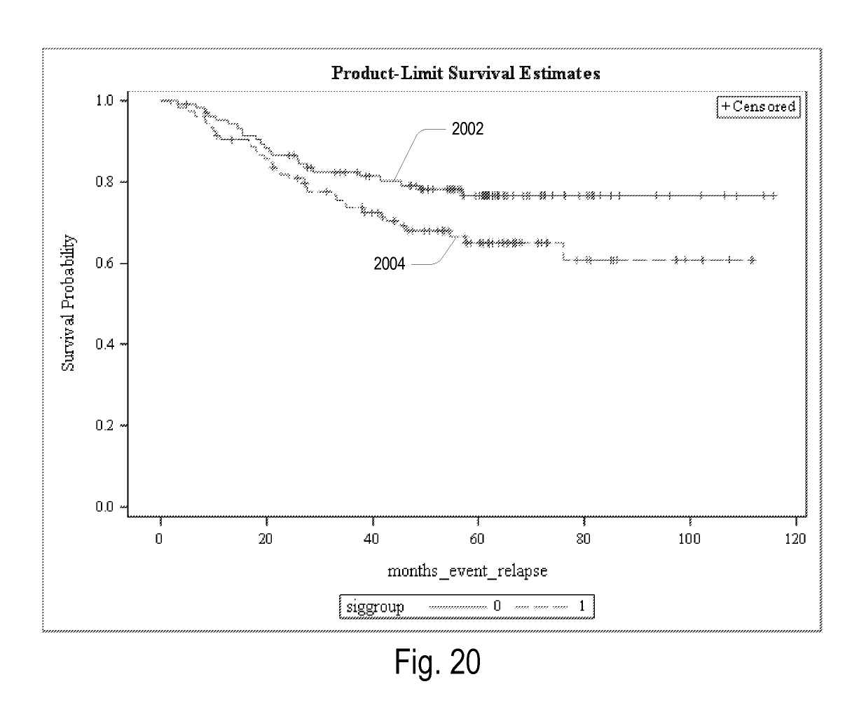

The hypoxic downregulated isoform of CARD8 was associated with poor prognosis in both cohorts. Patients having a top 50th percentile isoform ratio has a worse prognosis compared with patients having a bottom 50th percentile isoform ratio. FIG. 19 is a plot of the survival probability versus time for patients from the Lee cohort having a bottom 50th percentile isoform ratio (1902) and patients having a top 50th percentile isoform ratio (1904). FIG. 20 is a plot of the survival probability versus time for patients from the Okayama cohort having a bottom 50th percentile isoform ratio (2002) and patients having a top 50th percentile isoform ratio (2004).

What is claimed is:

1. A method of providing a prognosis to a subject suffering from an adenocarcinoma, the method comprising:
a) obtaining a biological sample from the subject;
b) measuring a relative expression level of one or more pairs of mRNA isoforms or downstream expression products thereof within the biological sample; and
c) comparing the relative expression level to relative historical or contemporary cohort expression levels of the one or more pairs of mRNA isoforms or downstream expression products thereof, the relative historical or contemporary cohort expression levels retrieved from a historical or contemporary data set for historical or contemporary patients suffering from the adenocarcinoma.

2. The method of claim 1, wherein at least one of the one or more pairs of mRNA isoforms or downstream expression products thereof is associated with a gene selected from the group consisting of ETNK1, CARD8, TMEM68, ATF71P, SUV420H1, PPM1B, and ZNF33A.

3. The method of claim 1, wherein at least one of the one or more pairs of mRNA isoforms or downstream expression products thereof is associated with a gene selected from the group consisting of ETNK1, CARD8, ATF71P, and PPM1B.

4. The method of claim 1, wherein the prognosis is a good prognosis if the relative expression level of a hypoxia upregulated isoform of ATF71P and a hypoxia downregulated isoform of ATF71P is increased and compared to the relative historical or contemporary cohort expression levels of the hypoxia upregulated isoform of ATF71P and the hypoxia downregulated isoform of ATF71P of the historical or contemporary data set.

5. The method of claim 1, wherein the prognosis is a poor prognosis if the relative expression level of a hypoxia upregulated isoform of ATF71P and a hypoxia downregulated isoform of ATF71P is decreased compared to the relative historical or contemporary cohort expression levels of the hypoxia upregulated isoform of ATF71P and the hypoxia downregulated isoform of ATF71P of the historical or contemporary data set.

6. The method of claim 1, wherein the prognosis is a good prognosis if the relative expression level of a hypoxia upregulated isoform of PPM1B and a hypoxia downregulated isoform of PPM1B is decreased compared to the relative historical or contemporary cohort expression levels of the hypoxia upregulated isoform of PPM1B and the hypoxia downregulated isoform of PPM1B of the historical or contemporary data set.

7. The method of claim 1, wherein the prognosis is a poor prognosis if the relative expression level of a hypoxia upregulated isoform of PPM1B and a hypoxia downregulated isoform of PPM1B is increased compared to the relative historical or contemporary cohort expression levels of the hypoxia upregulated isoform of PPM1B and the hypoxia downregulated isoform of PPM1B of the historical or contemporary data set.

8. The method of claim 1, wherein the prognosis is a good prognosis if the relative expression level of a hypoxia upregulated isoform of ETNK1 and a hypoxia downregulated isoform of ETNK1 is decreased compared to the relative historical or contemporary cohort expression levels of the hypoxia upregulated isoform of ETNK1 and the hypoxia downregulated isoform of ETNK1 of the historical or contemporary data set.

9. The method of claim 1, wherein the prognosis is a poor prognosis if the relative expression level of a hypoxia upregulated isoform of ETNK1 and a hypoxia downregulated isoform of ETNK1 is increased compared to the relative historical or contemporary cohort expression levels of the hypoxia upregulated isoform of ETNK1 and the hypoxia downregulated isoform of ETNK1 of the historical or contemporary data set.

10. The method of claim 1, wherein the prognosis is a good prognosis if the relative expression level of a hypoxia upregulated isoform of CARD8 and a hypoxia downregulated isoform of CARD8 is decreased compared to the relative historical or contemporary cohort expression levels of the hypoxia upregulated isform of CARD8 and the hypoxia downregulated isoform of CARD8 of the historical or contemporary data set.

11. The method of claim 1, wherein the prognosis is a poor prognosis if the relative expression level of a hypoxia upregulated isoform of CARD8 and a hypoxia downregulated isoform of CARD8 is increased compared to the relative historical or contemporary cohort expression levels of the hypoxia upregulated isoform of CARD8 and the hypoxia downregulated isoform of CARD8 of the historical or contemporary data set.

12. The method of claim 1, wherein the adenocarcinoma is adenocarcinoma of the esophagus, adenocarcinoma of the pancreas, adenocarcinoma of the prostate, adenocarcinoma of the cervix, adenocarcinoma of the stomach, adenocarcinoma of the breast, adenocarcinoma of the colon, adenocarcinoma of the small bowel or duodenum, adenocarcinoma of the lung, cholangiocarcinoma, adenocarcinoma of the vagina, adenocarcinoma of the ovary, adenocarcinoma of the salivary gland, adenocarcinoma of the uterus, adenocarcinoma of the cervix, or adenocarcinoma of the urachus.

13. The method of claim 1, wherein the subject is a mammal.

14. The method of claim 1, wherein the subject is a homo sapien.

15. The method of claim 1, wherein the biological sample comprises material selected from the group consisting of a tissue, a cell, a biopsy sample, blood, lymph, serum, plasma, urine, saliva, mucus, sweat, tears, and combinations thereof.

16. The method of claim 1, wherein the biological sample comprises blood.

17. The method of claim 1, the method further comprising:
d) treating the adenocarcinoma of the subject based on the outcome of step c).

18. The method of claim 17, wherein treating the adenocarcinoma comprises administering to the subject suffering from an adenocarcinoma a therapeutically effective amount of a compound selected from the group consisting of nimorazole, tirapazamine, TH302, SN30000, PR-104, AQ4N, and combinations thereof.

19-21. (canceled)

22. A composition of matter comprising an artificially synthesized polymerase chain reaction primer selected from the group consisting of SEQ ID NO: 15, SEQ ID NO. 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID No: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42.

23. A composition of matter comprising an artificially synthesized probe selected from the group consisting of SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, and SEQ ID NO: 56.
